(19) United States
(12) Patent Application Publication
(10) Pub. No.: US 2025/0163149 A1
(43) Pub. Date: May 22, 2025

(54) ANTI-RAGE ANTIBODY

(71) Applicant: Swansea University, Swansea (GB)

(72) Inventors: Robert Conlan, Swansea (GB); Deyarina Gonzalez, Swansea (GB)

(73) Assignee: Swansea University, Swansea (GB)

(21) Appl. No.: 18/842,364

(22) PCT Filed: Mar. 1, 2023
(86) PCT No.: PCT/GB2023/050461

§ 371 Date: Aug. 28, 2024

(30) Foreign Application Priority Data
Mar. 4, 2022 (GB) 2203066.2

(51) Int. Cl. C07K 16/28; A61K 47/68

(52) U.S. Cl. CPC C07K 16/2803; A61K 47/68031; A61K 47/6849; C07K 2317/24; C07K 2317/565; C07K 2317/77; C07K 2317/92

(57) ABSTRACT

The invention relates to an antibody, or an antigen binding fragment thereof, that selectively binds Receptor for Advanced Glycation Products (RAGE) and, ideally, provides for cell internalisation whereby said antibody can act as an antibody-drug-conjugate (ADC); a polynucleotide and/or vector encoding the said antibody; a cell transformed with said polynucleotide and/or said vector; a pharmaceutical composition comprising said antibody; an ADC comprising said antibody; and the use of said antibody, said ADC or said pharmaceutical composition in the treatment of or prevention of a disorder characterised by elevated RAGE expression, such as cancer.

### CROSS REFERENCE TO RELATED APPLICATIONS

This is the U.S. National Stage of International Application No. PCT/GB2023/050461, filed Mar. 1, 2023, which was published in English under PCT Article 21 (2), which in turn claims the benefit of Great Britain Application No. 2203066.2, filed Mar. 4, 2022. The PCT application is incorporated by reference in its entirety.

### FIELD OF THE INVENTION

The invention relates to an antibody, or an antigen binding fragment thereof, that selectively binds Receptor for Advanced Glycation Products (RAGE) and, ideally, provides for cell internalisation whereby said antibody can act as an antibody-drug-conjugate (ADC); a polynucleotide and/or vector encoding the said antibody; a cell transformed with said polynucleotide and/or said vector; a pharmaceutical composition comprising said antibody; an ADC comprising said antibody; and the use of said antibody, said ADC or said pharmaceutical composition in the treatment of or prevention of a disorder characterised by elevated RAGE expression, such as cancer.

Incorporation of Electronic Sequence Listing The Sequence Listing is submitted as an XML file named “Sequence.xml,” created on Aug. 27, 2024, 29,418 bytes, which is incorporated by reference herein.

### BACKGROUND OF THE INVENTION

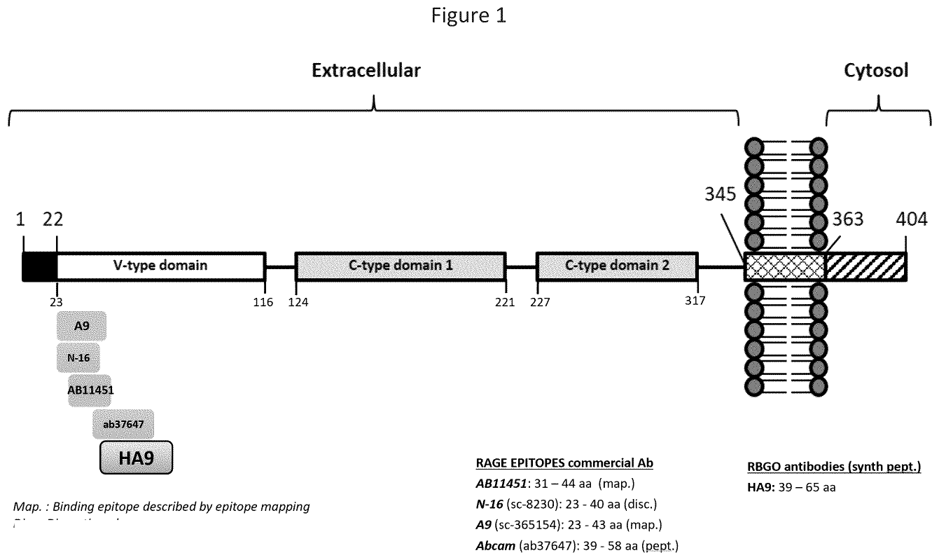

RAGE is a multiligand pattern recognition cell surface receptor that belongs to the immunoglobulin superfamily located on chromosome 6p21.3 at the major histocompatibility complex class III region. Full length RAGE protein is 404 amino acids, comprising an extracellular domain, a single hydrophobic transmembrane domain and a short cytosolic domain. Its ligand binding properties are provided by the extracellular domain, which can be divided into three functional regions; the V domain, C1 and C2 domains (FIG. 1). An increasing number of ligands are known to bind RAGE including, advanced glycation end products (the receptors namesake), high-mobility group protein 1, and members of the S100 protein family. RAGE binds a variety of damage-associated molecules, including glycated proteins, lipids and DNA, as well as pathogen-associated molecules from bacteria, viruses and parasites, and upon activation, it elicits pro-inflammatory processes.

Central to its role in an inflammatory response, is the internalisation of RAGE following ligand binding, this internalisation is a key component of RAGE-mediated signal transduction. The tissue distribution of RAGE under physiological conditions is limited, and with the exception of the lungs, expression is low. However, in certain disease conditions, up-regulation of RAGE expression is observed, in particular in a range of inflammatory diseases such as diabetes, sepsis and Alzheimer's disease. There is also evidence linking RAGE to cancer progression in mice and humans. Accordingly, its differential expression during disease therefore presents an opportunity to selective target this molecule for treating certain diseases, via the use of antibody-drug conjugation.

We have previously shown that RAGE is a suitable target for ADC therapy in colorectal and endometrial cancers, which are sensitive to an ADC treatment linked to cytotoxic drug (WO2016063060). Indeed, RAGE-targeting ADCs were up to 100-fold more efficacious in these cancer cells compared to non-malignant cells and up to 200-fold more cytotoxic than drug treatment alone.

However, key to the development of ADCs is the optimization of each constituent part i.e., the drug and the targeting antibody molecule. We have noted from preliminary studies a variability in the internalization of different RAGE antibodies, leading to a variation in effective cell killing. Accordingly, since these studies, we have further explored the binding epitope of RAGE antibodies. Our epitope mapping experiments have defined the epitope of the anti-RAGE antibody and coupled with our genetic screening of cancer and healthy patients we have confirmed the epitope sequence is highly stable (with 96% sequence identity), indicating a high level of conservation of the epitope coding sequence. We have therefore utilised the binding epitope to further elucidate antibodies with highest efficacy in terms of binding/internalisation and delivery of cargo for targeted cell killing.

Herein we disclose an optimised anti-RAGE antibody (hereinafter called HA9) for RAGE-targeted therapeutics. Despite the finding that this preferred antibody has weaker binding affinity, compared to known RAGE antibodies including those binding the same or overlapping epitopes, unexpectedly we have found that this antibody was rapidly and significantly internalised into RAGE expressing cells, even when compared to other antibodies binding similar epitopes on the RAGE protein. This unique antibody paves the way for improved therapeutic delivery of RAGE antibody associated cargo, such as a therapeutic agent, into RAGE expressing cells, particularly various cancers cells wherein RAGE is known to be overexpressed, permitting increased drug delivery and/or superior targeted cell killing. In addition, the antibody was found to have high specificity for cancer cells overexpressing RAGE with little or no cross-reactivity/binding with healthy cells when compared to other anti-RAGE antibodies, and also exhibiting reduced toxicity, providing for an improved anti-RAGE antibody therapeutic.

### STATEMENTS OF INVENTION

According to a first aspect of the invention there is therefore provided an isolated antibody, or an antigen binding fragment thereof, comprising: at least one complementarity-determining region, including any combination thereof, selected from the group comprising or consisting of:

i)
CDR1:
(SEQ ID NO: 1)
DHWMN;
ii)
CDR2:
(SEQ ID NO: 2)
QIRNRPYKFEKYYSDSVKG;
iii)
CDR3:
(SEQ ID NO: 3)
YRYGLAY;
iv)
CDR4:
(SEQ ID NO: 4)
RASQTIDTRIH;
v)
CDR5:
(SEQ ID NO: 5)
YISESLS;
and
vi)
CDR6:
(SEQ ID NO: 6)
QQTESWPIT;

or
vii) an amino acid sequence having at least 70% sequence similarity with, or identity with, one of the sequences of parts i) to vi).

In a preferred embodiment of the invention said antibody or fragment includes at least the CDR: v) CDR5: YISESLS (SEQ ID NO:5).

The term “antibody” used herein refers to an immunoglobulin protein having two “heavy chain” subunits and two smaller “light chain” subunits. The four subunits are assembled to form two Fab (fragment antigen-binding) regions that each recognize and bind to a particular antigen (e.g. Receptor for Advanced Glycation End Products, or RAGE) and one Fc (fragment, crystallisable) region. Each Fab region is made from one heavy chain and one light chain, which each contributes one constant domain and one variable domain. Each Fc region is made from two heavy chains, which each contribute two or three constant domains.

The antibody described herein may be any class of antibody, for example IgA, IgD, IgE, IgG, or IgM antibody. In particular, the antibody described herein may be IgD, IgE, or IgG antibody. Preferably, the antibody is an IgG antibody.

In a preferred embodiment, the antibody is a monoclonal antibody.

Reference herein to a complementarity-determining region is reference to a part of said antibody that binds with the antibody's target and is characterized by one of the specified amino acid sequences i)-vi) or vii).

In yet a further preferred embodiment of the invention said amino acid sequence of part vii) has 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence similarity with, or identity with, one of the sequences in parts i)-vi).

In a preferred embodiment of the invention said antibody has a light chain variable region and a heavy chain variable region, wherein at least one of the complementarity determining regions of the heavy chain variable region and at least one of the complementarity determining regions of the light chain variable region have the following amino acid sequence:

heavy chain:
CDR1:
(SEQ ID NO: 1)
DHWMN,
CDR2:
(SEQ ID NO: 2)
QIRNRPYKFEKYYSDSVKG
or
CDR3:
(SEQ ID NO: 3)
YRYGLAY;
light chain:
CDR4:
(SEQ ID NO: 4)
RASQTIDTRIH,
CDR5:
(SEQ ID NO: 5)
YISESLS
or
CDR6:
(SEQ ID NO: 6)
QQTESWPIT;

or an amino acid sequence having at least 70% sequence similarity or identity thereto, or more preferably having 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence similarity or identity thereto.

In yet a further preferred embodiment the heavy chain variable region comprises a plurality, including any combination, of the following sequences, including all of the following sequences CDR1: DHWMN (SEQ ID NO:1), CDR2: QIRNRPYKFEKYYSDSVKG (SEQ ID NO:2) or CDR3: YRYGLAY (SEQ ID NO:3); or an amino acid sequence having at least 70% sequence similarity or identity thereto, or more preferably having 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence similarity or identity thereto.

In yet a further preferred embodiment the light chain variable region comprises a plurality, including any combination, of the following sequences, including all of the following sequences CDR4: RASQTIDTRIH (SEQ ID NO:4), CDR5: YISESLS (SEQ ID NO:5) or CDR6: QQTESWPIT (SEQ ID NO:6), or an amino acid sequence having at least 70% sequence similarity or identity thereto, or more preferably having 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence similarity or identity thereto.

In yet a more particular embodiment of the invention said antibody comprises all of the following complementarity determining regions: CDR1: DHWMN (SEQ ID NO:1), CDR2: QIRNRPYKFEKYYSDSVKG (SEQ ID NO:2), CDR3: YRYGLAY (SEQ ID NO:3), CDR4: RASQTIDTRIH (SEQ ID NO:4), CDR5: YISESLS (SEQ ID NO:5) and CDR6: QQTESWPIT (SEQ ID NO:6).

In the exemplary antibody, HA9, the heavy chain variable region comprises or consists of the amino acid sequence of:

(SEQ ID NO: 7)
EVKLVETGGGLVQPGRPMKLSCVASGFTLSDHWMNWVRQSP
EKGLEWVAQIRNRPYKFEKYYSDSVKGRFTISRDDSKNSVY
LQMNNLRAEDMGIYYCSSYRYGLAYWGQGTLVTVST
(CDRs underlined);

or an amino acid sequence having at least 70% sequence similarity or identity thereto; and/or
the light chain variable region comprises or consists of the amino acid sequence:

(SEQ ID NO: 8)
DNLLTQSPAILSVSPGERVSFSCRASQTIDTRIHWYQQRKN
GSPRLLIRYISESLSGIPSRFSGSGSGTEFTLTINSVESED
IADYYCQQTESWPITFGSGTKLEIK
(CDRs underlined);

or an amino acid sequence having at least 70% sequence similarity or identity thereto.

In yet a further preferred embodiment of the invention said heavy chain variable region or light chain variable region has an amino acid sequence having 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence similarity with, or identity with,

(SEQ ID NO: 7)
EVKLVETGGGLVQPGRPMKLSCVASGFTLSDHWMNWVRQSP
EKGLEWVAQIRNRPYKFEKYYSDSVKGRFTISRDDSKNSVY
LQMNNLRAEDMGIYYCSSYRYGLAYWGQGTLVTVST
(CDRs underlined);
or
(SEQ ID NO: 8)
DNLLTQSPAILSVSPGERVSFSCRASQTIDTRIHWYQQRKN
GSPRLLIRYISESLSGIPSRFSGSGSGTEFTLTINSVESED
IADYYCQQTESWPITFGSGTKLEIK
(CDRs underlined),

respectively.

As is known, the ability of an antibody to recognize and bind to its antigen is determined by its CDRs, and that other variations in the sequences of the heavy chain and light chains do not significantly affect the binding ability of the antibody, it is therefore expected that antibodies having the same CDR sequences as the HA9 antibody disclosed herein, but with other variations in the heavy chain and/or light chains will have substantially the same binding properties as the HA9 antibody. Accordingly, the invention extends to embodiments including such variations.

In yet a more particular preferred embodiment of the invention said antibody is a human antibody or a humanized antibody or an antibody fragment thereof. In a humanized antibody, the CDRs may be derived from a rat or mouse monoclonal antibody, while the framework of the variable regions and the constant regions of the antibody may be derived from a human antibody or modified to more closely resemble an antibody produced by a human. Such a humanized antibody elicits a negligible immune response when administered to a human compared to the immune response mounted by a human against a rat or mouse antibody. Methods of preparing humanized antibodies are known in the art.

The antibody or antigen biding fragment may, for example, be a chimeric antibody or antigen binding fragment. Chimeric antibodies or antigen binding fragments are those containing amino acid sequences from two or more species. Chimeric antibodies or antigen binding fragments are often made to make the antibody or antigen binding fragment more like a human antibody or antigen binding fragment but do not always fall within the definition of humanized antibodies or antigen binding fragments. For example, the antibody or antigen binding fragments may have a constant region from a human antibody and a variable region from a non-human antibody (e.g. a mouse or rat antibody).

In some examples, the antibody or antigen binding fragment disclosed herein is a recombinant antibody or antigen binding fragment. Recombinant antibodies or antigen binding fragments are antibodies or antigen binding fragments that have been produced using recombinant antibody coding genes.

The term “antigen binding fragment” used herein refers to a protein that comprises only part of the structure of an antibody but includes at least one region (a complementarity determining region) that recognizes and binds to a particular antigen (e.g. RAGE).

The antigen binding fragment may, for example, comprise, or consist of one or more Fab region(s) (each made from one heavy chain and one light chain, which each contributes one constant domain and one variable domain). For example, the antigen binding fragments may comprise or consist of one Fab region or the antigen binding fragment may comprise or consist of two Fab regions (F (ab) 2).

The antigen binding fragment may, for example, comprise or consist of one or more light chain variable regions and one or more heavy chain variable regions. The light chain variable region and heavy chain variable region may, for example, be part of the same polypeptide and may, for example, be joined by one or more flexible linker sequence(s). The antigen binding fragment may, for example, comprise or consist of more than one light chain variable region and/or more than one heavy chain variable region which may, for example, be part of the same polypeptide and may, for example, be joined by one or more flexible linker sequence(s). An antigen binding fragment comprising or consisting of a light chain variable region and a heavy chain variable region on a single polypeptide may, for example, be referred to as a single-chain variable fragment (scFv). The scFvs may, for example, be engineered to non-covalently bind to other scFvs (which may be the same or different) to form bivalent molecules referred to as diabodies. The scFvs may, for example, comprise more than one light chain variable regions and/or more than one heavy chain variable regions on the same polypeptide (e.g. more than one scFv on the same polypeptide) and be referred to as tandem scFvs.

The antigen binding fragment may, for example, comprise or consist of a single heavy chain (including constant and variable domains) and a single light chain (including constant and variable domains). The constant domains of the heavy chain and the light chain may each independently be complete or truncated.

The antigen binding fragment may, for example, comprise or consist of two heavy chains (both including constant and variable domains) and two light chains (both including constant and variable domains) where the constant domains of one or more of the heavy and light chains are truncated.

The antigen binding fragment may, for example, comprise or consist of a single monomeric variable domain. These antigen binding fragment may, for example, be referred to as nanobodies. The antigen binding fragment may, for example, comprise or consist of a single heavy chain including one or more constant domains and one variable domain. The antigen binding fragment may, for example, comprise or consist of a single heavy chain including two constant domains and one variable domain.

In yet a further preferred embodiment of the invention said antibody, or antigen binding fragment thereof, binds specifically to RAGE, and ideally, human RAGE (Uniprot accession number: Q15109-1), including any or all epitopes thereof.

As is known in the art, full length RAGE comprises an extracellular domain, a single hydrophobic transmembrane domain and a short cytosolic tail, with the extracellular domain, further divided into three functional regions; the V domain, C1 and C2 domains (FIG. 1). The antibody or antibody fragments of the present invention bind to an epitope having the sequence AQNITARIGEPLVLKCKGAPKKPPQRLEWKLNTGRT (SEQ ID NO:9) of the V-domain, or an epitope with an amino acid sequence that is at least about 90% similar to or identical or to the corresponding epitope having the sequence AQNITARIGEPLVLKCKGAPKKPPQRLEWKLNTGRT (SEQ ID NO:9), or more ideally an epitope that is at least about 91% or at least about 92% or at least about 93% or at least about 94% or at least about 95% or at least about 96% or at least about 97% or at least about 98% or at least about 99% 100% identical to the corresponding epitope having the sequence

(SEQ ID NO: 9)
AQNITARIGEPLVLKCKGAPKKPPQRLEWKLNTGRT

Amino acid sequences with a degree of similarity or identity may be determined using the BLAST® (Basic Local Alignment Search Tool) provided by National Center for Biotechnology Information (NCBI).

The antibody and antigen binding fragment(s) of the present invention bind RAGE, in isolation or when expressed on the cell surface and in the latter case are advantageously internalised into cells. Through such internalisation, the identification of cells with expression of RAGE, e.g. for imaging or diagnosis, or therapy, e.g. via the selective delivery of therapeutic agent into RAGE expressing cells, can be achieved.

Binding RAGE can be determined by different techniques including, but not limited to, Surface Plasmon Resonance (Biacore), to evaluate the characteristics of the interaction of RAGE antibodies in isolation with its ligands (in this case, recombinant protein or synthetic peptides), or by immunoblotting techniques, to identify the presence of accessible RAGE protein in protein extracts or on the cell surface of cells (including immunofluorescence or western blot).

Internalisation of antibodies into RAGE expressing cells can be determined by assays such as, but not limited to, internalization assay (in vitro) using direct or indirect immunofluorescence to detect and quantify amount of RAGE antibody taken up by cells, or by biodistribution study (in vivo) using fluorescently labelled RAGE antibodies administered into animal models to detect accumulation in RAGE+tissues.

The antibody and antigen binding fragment of the present invention may have a binding affinity constant (KD) for RAGE equal to or less than about 5.65×10⁹ M

The antibody or antigen binding fragment(s) may, for example, be modified to include one or more labels. The labels may, for example, be radioactive labels, fluorescent labels, luminescent labels, biotin labels, and enzymatic labels.

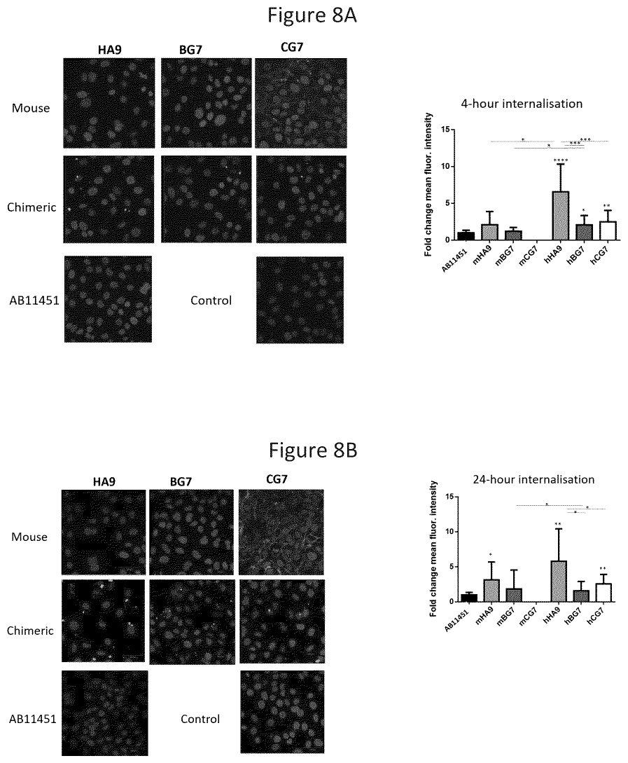

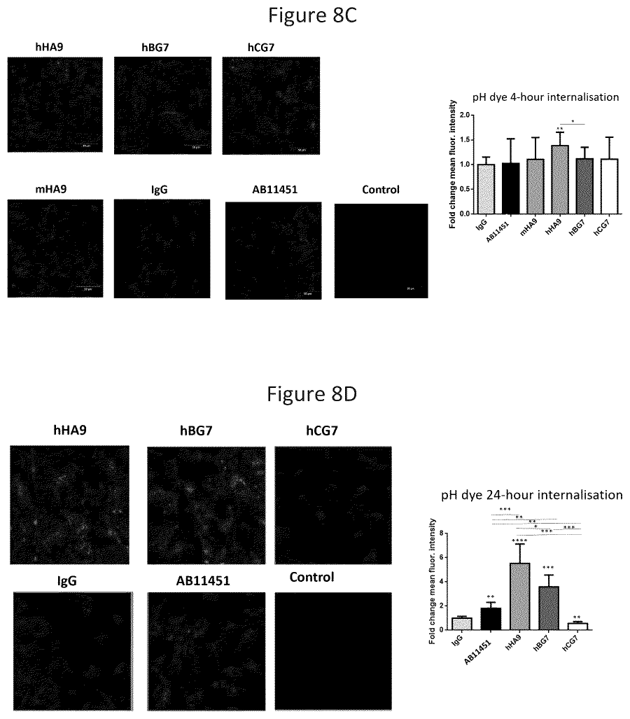

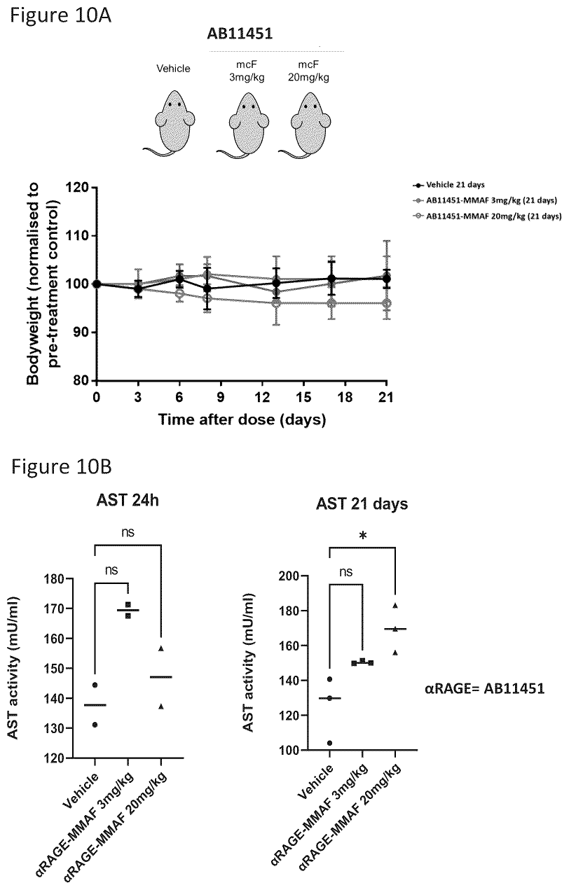

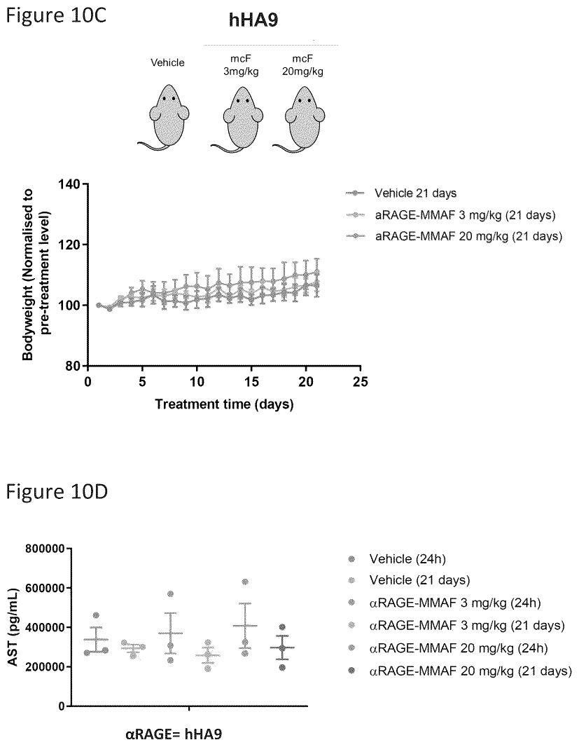

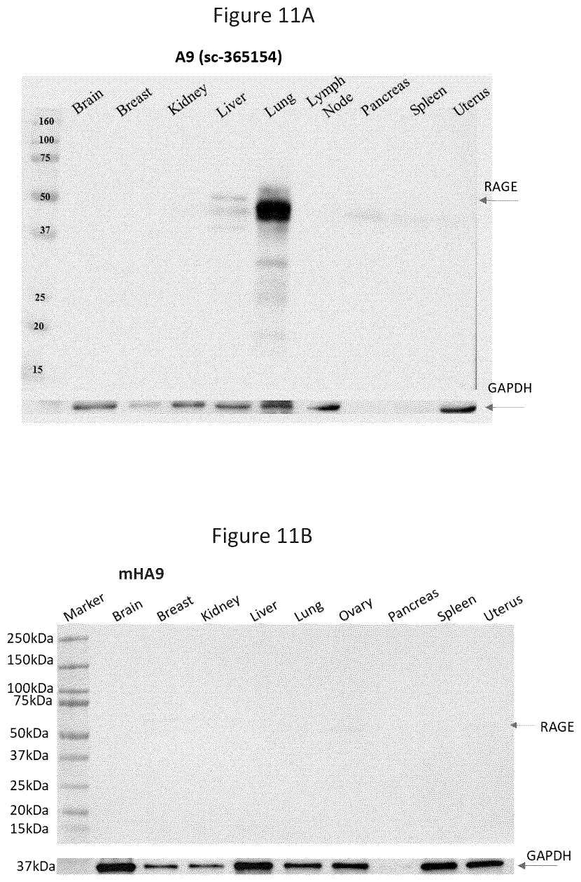

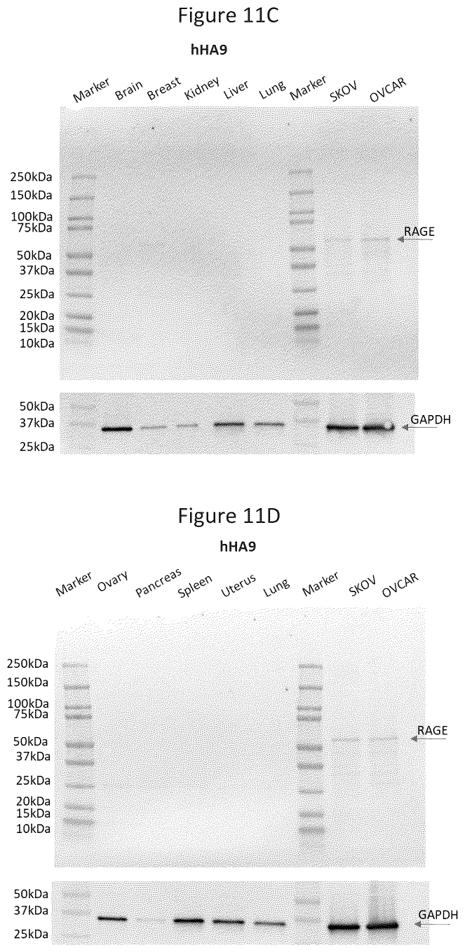

We have devised a superior anti-RAGE antibody for the development of a novel anti-RAGE cell targeted therapeutic strategies. The antibody defined herein, whilst exhibiting weaker binding affinity compared to known RAGE antibodies that bind a similar V-domain epitope, has shown, unexpectedly, rapid and increased internalisation, even when compared to alternative antibodies raised against the same epitope (FIGS. 8A-8D). Without wishing to be bound by theory, we consider the precise binding sequence provides for both targeted delivery and internalisation into the cell. As will be known to those skilled in the art, conjugation of the claimed antibody, or its fragment(s), to or with associated therapeutic agents, will permit more rapid and increased delivery of said agent into the cell (and, in the context of cytotoxic agents, increased cell killing). Additionally, the superior anti-RAGE antibody (hHA9) exhibited a safer toxicity profile defined here by the levels of the aspartate aminotransferase enzyme (AST) released by the liver into the bloodstream (FIG. 10D); which remained at similar levels for all treatments groups for the duration of the study. On the contrary AB11451 ADC formulations of similar DAR and payload exhibited elevated levels of AST, indicative of liver damage (FIG. 10B). Without wishing to bound by theory, we have reason to believe that this is due to the fact that HA9 was also observed to have little or no expression in healthy tissues when compared to other available antibodies (FIGS. 11A-11D).

According to a further aspect of the invention there is provided one or more polynucleotides encoding the isolated antibody or said antigen binding fragment thereof as disclosed herein.

Thus, the polynucleotides encode one or more or all of the CDRs, or the heavy chain variable region, or the light chain variable region, or the heavy chain constant region, or the light chain constant region, or the heavy chain, or the light chain, of the antibody disclosed herein. The polynucleotides may, for example, be a DNA or RNA molecule.

According to a yet further aspect of the invention there is provided one or more vectors comprising the one or more polynucleotides as disclosed herein.

The vectors preferably include elements which allow expression of said polynucleotides in a selected host cell. The vectors are, for example plasmid or viral vectors, and are useful for transforming host cells in order to clone or express the said antibody or polynucleotides disclosed herein. The vector usually includes a promoter, signals for initiation and termination of translation, as well as appropriate regions of regulation of transcription. The vector may optionally possess particular signals specifying the secretion of the translated protein. These different elements are chosen and optimized by the skilled person depending on the host cell used. Such vectors are prepared by methods commonly used by those skilled in the art, and the resulting clones can be introduced into an appropriate host by standard methods, such as lipofection, electroporation, heat shock, or chemical methods.

According to another aspect of the invention there is provided at least one host cell comprising the one or more polynucleotides as disclosed herein, or the one or more vectors as disclosed herein. The cell may, for example, be referred to as a recombinant and/or host cell. The cell may, for example, be an established cell line (a cell that demonstrates the potential for indefinite subculture in vitro).

The cell may, for example, be a hybridoma. The cell may, for example, be prokaryotic (e.g. Escherichia coli) or eukaryotic (e.g. protist cell, animal cell (e.g. mammalian cell such as CHO or COS or HEK 293 cells, avian cell, insect cell such as Sf9 cell), plant cell, fungal cell (e.g. yeast cell such as Saccharomyces cerevisiae)).

In accordance with a further aspect of the present invention there is provided a pharmaceutical composition comprising the said antibody or antigen binding fragment(s) described herein and a pharmaceutically acceptable carrier, excipient, or diluent. Preferably, said pharmaceutical composition comprises a pharmaceutically effective amount of said antibody and/or said antigen binding fragment thereof.

Suitable pharmaceutical excipients are well known to those of skill in the art. Pharmaceutical compositions may be formulated for administration by any suitable route, for example oral, rectal, nasal, bronchial (inhaled), topical (including eye drops, buccal and sublingual), vaginal or parenteral (including subcutaneous, intramuscular, intravenous and intradermal) administration and may be prepared by any methods well known in the art of pharmacy.

The composition may be prepared by bringing into association the said antibody or antigen binding fragment(s) as defined herein with the carrier. In general, the formulations are prepared by uniformly and intimately bringing into association the said antibody or antigen binding fragment(s) with liquid carriers or finely divided solid carriers or both and then, if necessary, shaping the product. The invention extends to methods for preparing a pharmaceutical composition comprising bringing the said antibody or antigen binding fragment(s), as defined herein together in conjunction or association with a pharmaceutically or veterinary acceptable carrier or vehicle.

Parenteral formulations will generally be sterile.

For topical application to the skin, the composition may be made up into a cream, ointment, jelly, solution or suspension etc. Cream or ointment formulations that may be used for the drug are conventional formulations well known in the art, for example, as described in standard textbooks of pharmaceutics such as the British Pharmacopoeia.

The precise amount of a composition as defined herein which is therapeutically effective, and the route by which such compound is best administered, is readily determined by one of ordinary skill in the art. Such amounts will depend, of course, on the particular condition being treated, the severity of the condition, the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for any other reasons. Other factors include the desired period of treatment. In the event that a response in a subject is insufficient at the initial doses applied, higher doses (or effectively higher doses by a different, more localized delivery route) may be employed to the extent that patient tolerance permits.

According to a further aspect of the invention there is provided a combination therapeutic comprising the antibody or antigen binding fragment as defined herein in combination with at least one other therapeutic agent.

Ideally, said at least one other therapeutic agent is an agent used to treat or prevent cancer or its associated symptoms.

According to a further aspect of the invention there is provided said antibody, or antigen binding fragment(s), or said pharmaceutical composition or said combination therapeutic for use in the treatment, or prevention, of disorders characterised by elevated RAGE expression such as, but not limited to, diabetes, neurological disorders and cancer, including, but not limited to, specific cancers as ovarian cancer, endometrial cancer, breast cancer and prostate cancer.

According to a yet further aspect of the invention there is provided the use of said antibody, or antigen binding fragment(s), or said pharmaceutical composition or said combination therapeutic in the manufacture of a medicament to treat, or prevent, disorders characterised by elevated RAGE expression such as, but not limited to, diabetes, neurological disorders or cancer, including, but not limited to, specific cancers as ovarian cancer, endometrial cancer, breast cancer and prostate cancer.

According to a further aspect of the invention there is provided a method of treating a disorder characterised by elevated RAGE expression comprising administering said antibody, or antigen binding fragment(s), or said pharmaceutical composition or said combination therapeutic to an individual.

In certain embodiments, the subject is a human. In other embodiments, the subject is a mammal other than a human, such as non-human primates (e.g. apes, monkeys and lemurs), companion animals such as cats or dogs, working and sporting animals such as dogs, horses and ponies, farm animals such as pigs, sheep, goats, deer, oxen and cattle, and laboratory animals such as rodents (e.g. rabbits, rats, mice, hamsters, gerbils or guinea pigs).

In accordance with a further aspect of the present invention there is provided the non-therapeutic (e.g., in vitro) uses of the antibodies and antigen binding fragment(s) of the present invention. For example, the antibodies and antigen binding fragment(s) of the invention may be used for diagnosing, detecting, monitoring, a disease.

Preferred features of each aspect of the invention may be as described in connection with any of the other aspects.

Throughout the description and claims of this specification, the words “comprise” and “contain” and variations of the words, for example “comprising” and “comprises”, mean “including but not limited to” and do not exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

All references, including any patent or patent application, cited in this specification are hereby incorporated by reference. No admission is made that any reference constitutes prior art. Further, no admission is made that any of the prior art constitutes part of the common general knowledge in the art.

Other features of the present invention will become apparent from the following examples. Generally speaking, the invention extends to any novel one, or any novel combination, of the features disclosed in this specification (including the accompanying claims and drawings). Thus, features, integers, characteristics, compounds or chemical moieties described in conjunction with a particular aspect, embodiment or example of the invention are to be understood to be applicable to any other aspect, embodiment or example described herein, unless incompatible therewith.

Moreover, unless stated otherwise, any feature disclosed herein may be replaced by an alternative feature serving the same or a similar purpose.

The Invention will now be described by way of example only with reference to the Examples below and to the following Figures wherein:

FIG. 1: Schematic of human RAGE protein domains and approximate binding epitopes of various RAGE antibodies

Schematic representation of full-length human RAGE protein and its different protein domains including (from left to right): the signal peptide, an extracellular region composed by 3 immunoglobulin (Ig)-like domains (one V-type domain and two C-type domains, C1 and C2), a single transmembrane helix and a short intracellular cytosolic domain. Numbers indicate first and last amino acid (aa) of each domain based on the canonical sequence for full length RAGE protein (Uniprot accession number Q15109-1). The following proprietary/commercial anti-RAGE antibodies, the description of their binding epitopes on RAGE V-domain and the reference numbers in the case of the commercial antibodies have been included in the figure: HA9 (39-65 aa), AB11451 (31-44 aa), A9 (sc-365154, 23-43 aa), N-16 (sc-8230, 23-40 aa), Abcam (ab37647, 39-58 aa).

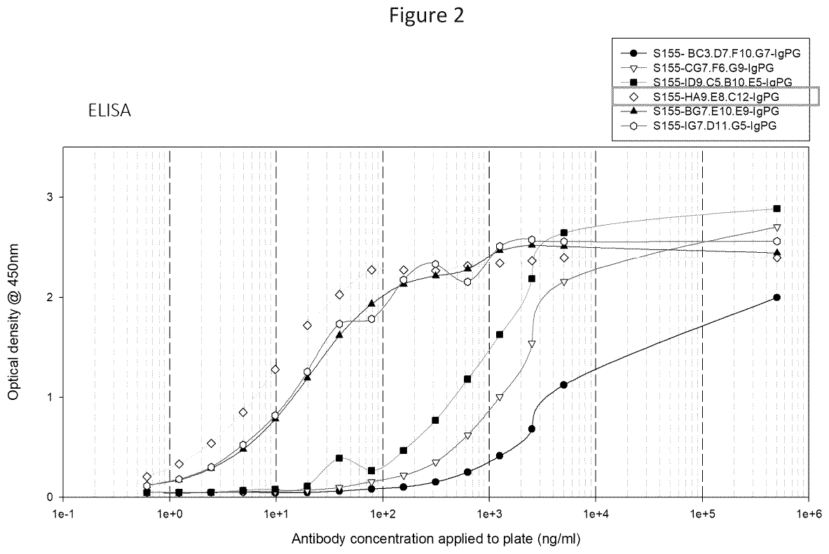

FIG. 2: Screening of purified anti-RAGE antibodies by enzyme linked immunosorbent assay (ELISA). BSA-conjugated synthetic peptides of RAGE protein were immobilized onto microplates and incubated with two-fold serial dilutions of antibodies. The graph shows the binding curves of 6 purified anti-RAGE antibody clones to immobilised RAGE protein synthetic peptide. The HA9 clone (highlighted in the legend) is the antibody displaying the best binding response to the antigen.

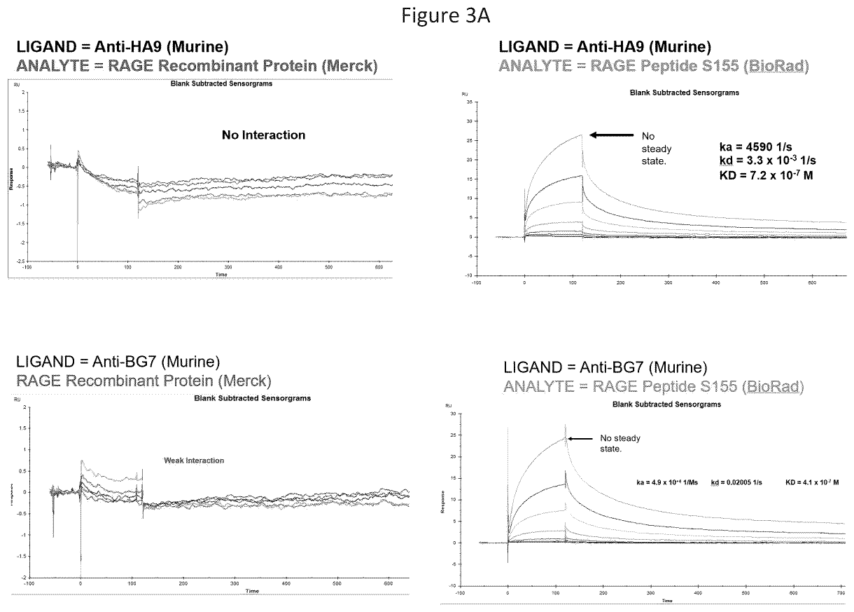

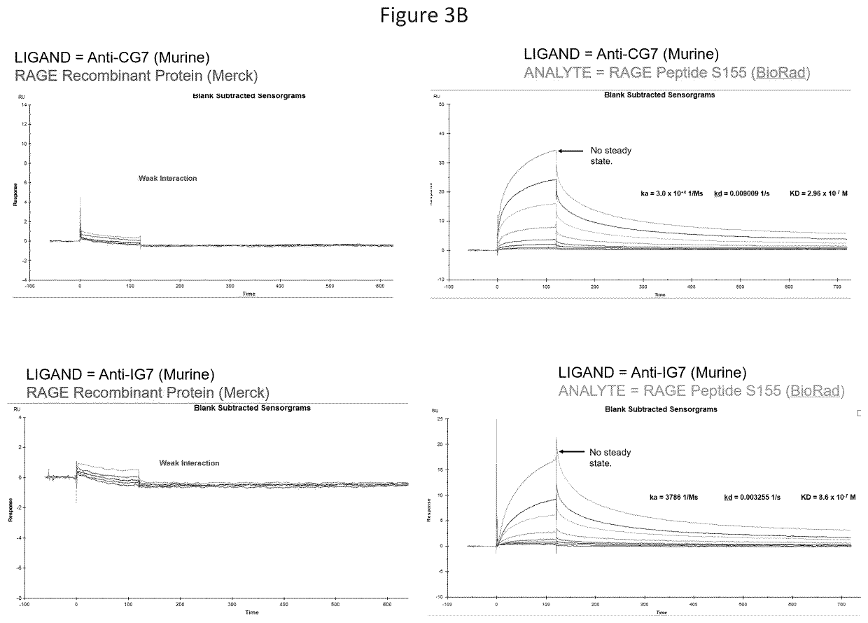

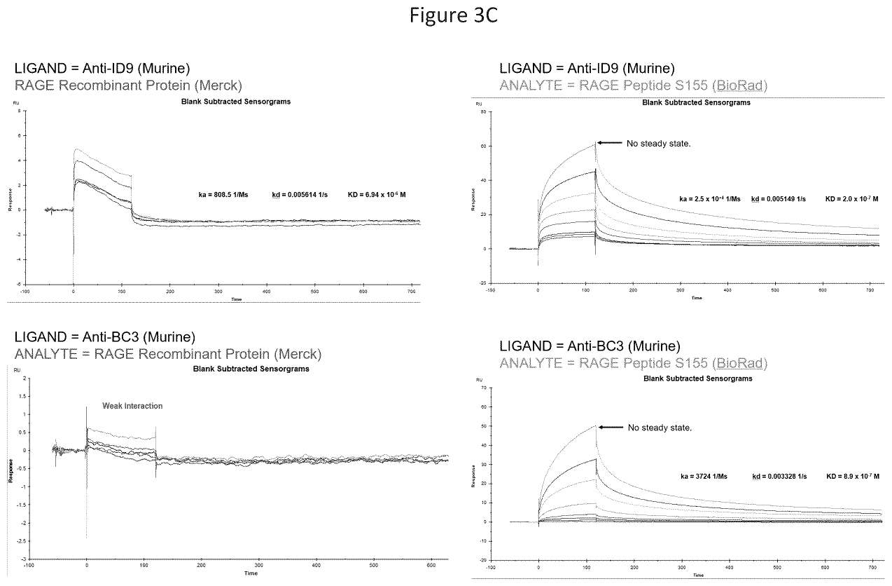

FIGS. 3A-3C: Binding kinetics between murine anti-RAGE antibody clones and RAGE recombinant protein, or RAGE synthetic peptide, by surface plasmon resonance show weak affinity interaction for mHA9 clone. Ligands (murine antibodies) were captured to a Sensor Chip CM5 via amine coupling method followed by injection of analytes (recombinant protein or synthetic peptide) using a multi-cycle kinetics approach with five-seven concentration range. Data were then fitted to a 1:1 binding model. The ligand and analyte used in each binding kinetic assay are described at the top of each displayed sensorgram.

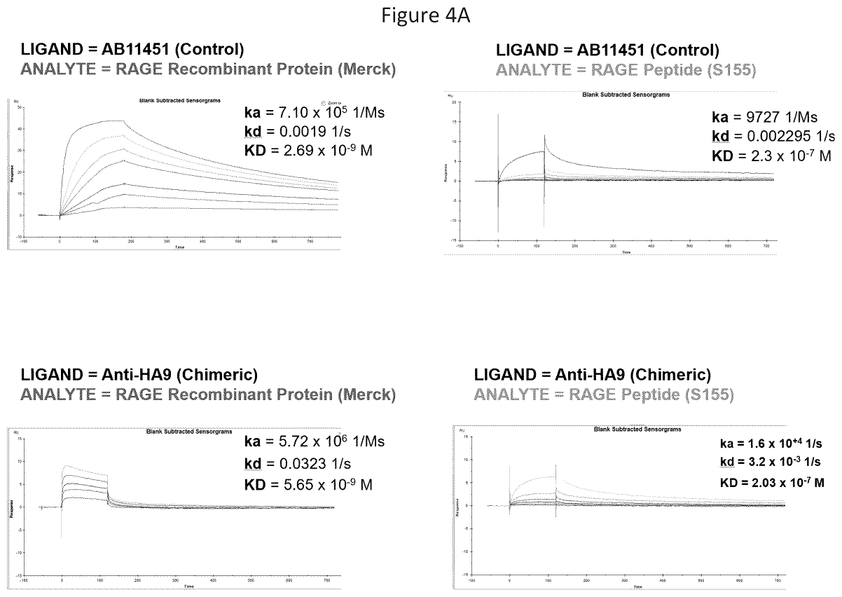

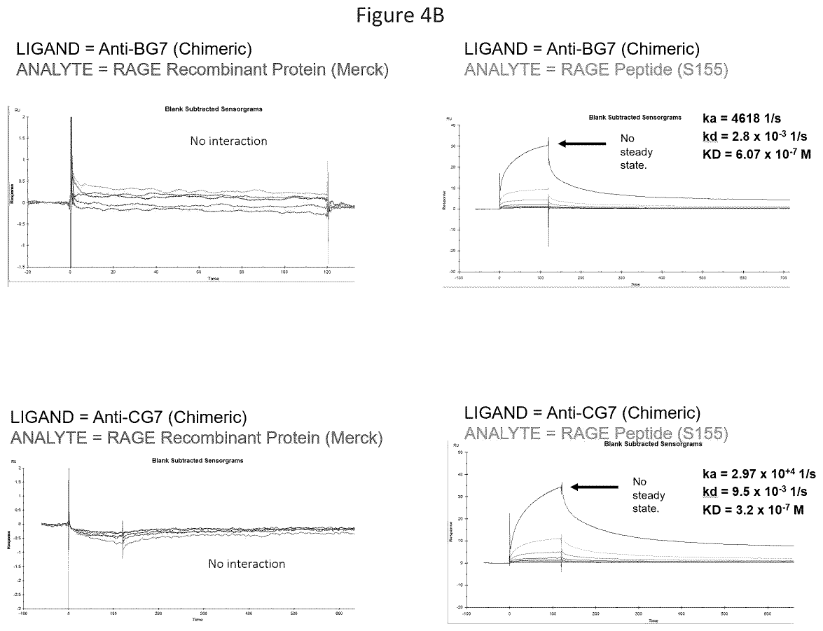

FIGS. 4A-4B: Binding kinetics between chimeric anti-RAGE antibody clones, or AB11451 antibody, and RAGE recombinant protein, or RAGE synthetic peptide, by surface plasmon resonance results in high affinity interaction for AB11451 and weaker affinity for hHA9 clone. Ligands (chimeric antibodies or AB11451) were captured to a Sensor Chip CM5 via amine coupling method followed by injection of analytes (recombinant protein or synthetic peptide) using a multi-cycle kinetics approach with five-seven concentration range. Data were then fitted to a 1:1 binding model. The ligand and analyte used in each binding kinetic assay are described at the top of each displayed sensorgram.

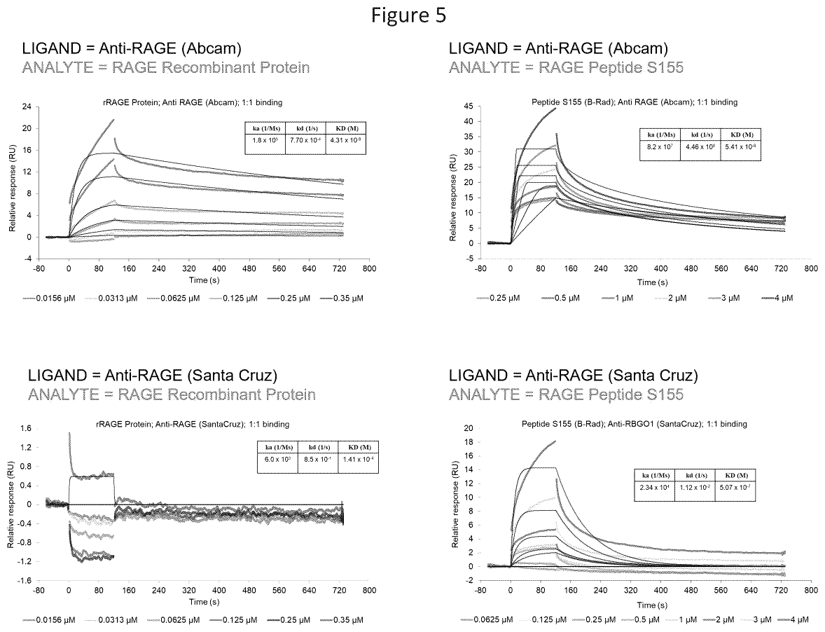

FIG. 5: Binding kinetics between commercial anti-RAGE antibodies (including Abcam ab37647 and Santa Cruz sc-365154) and RAGE recombinant protein, or RAGE synthetic peptide, by surface plasmon resonance shows high affinity interaction. Ligands (commercial antibodies) were captured to a Sensor Chip CM5 via amine coupling method followed by injection of analytes (recombinant proteins or synthetic peptides) using a multi-cycle kinetics approach with five-seven concentration range. Data were then fitted to a 1:1 binding model. The ligand and analyte used in each binding kinetic assay are described at the top of each displayed sensorgram.

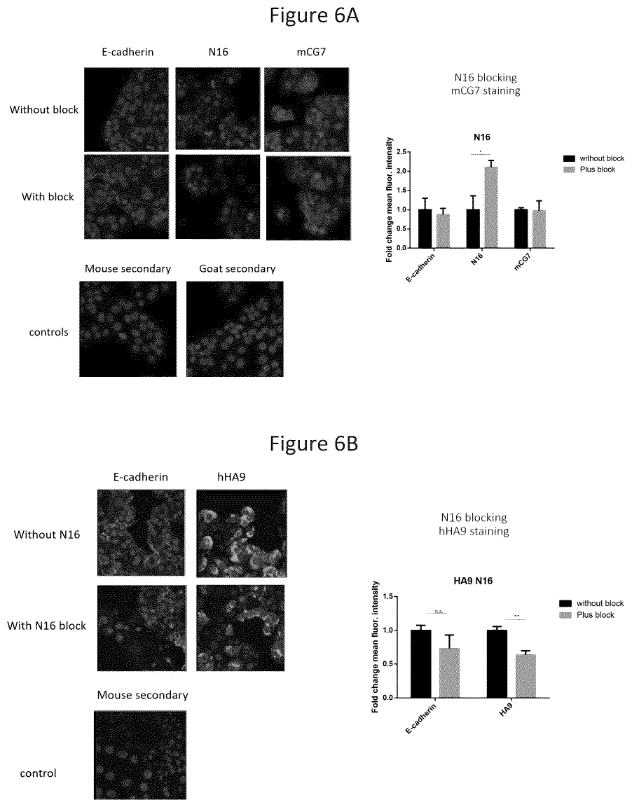

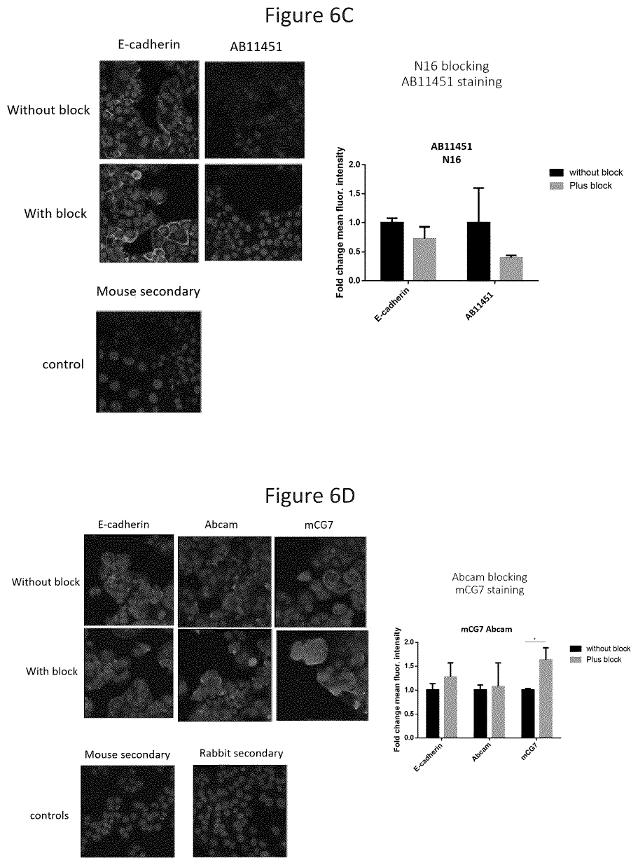

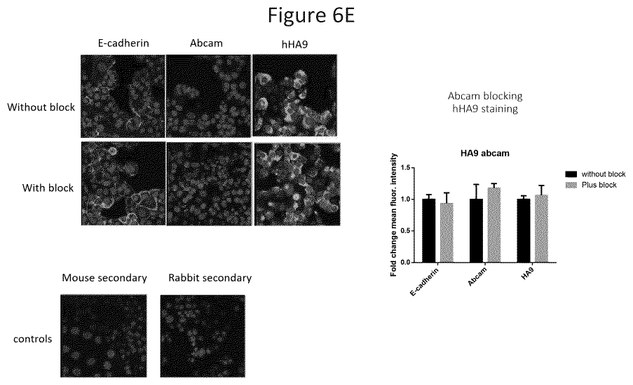

FIGS. 6A-6E: Binding target blocking assay suggests N16 and hHA9 antibodies share similar binding epitopes. HEC-1A cells were fixed/permeabilized/blocked and pre-incubated with a primary antibody against RAGE at a concentration in excess to saturate its specific binding epitope followed by incubation with the α-RAGE antibody of interest, or E-cadherin as a negative control antibody. The binding of the latest were detected with a fluorescent conjugate secondary antibody. Secondary antibody-only staining were also included as negative controls. Nuclei were counterstained with Hoechst. Images were acquired on a Zeiss LSM 710 confocal microscope and mean fluorescent intensity per field was quantified as a function of number of cells using ImageJ analysis software. Left panels show representative confocal images obtained with/without previous N-16 antibody blocking (FIGS. 6A, 6B, 6C) or Abcam antibody blocking (FIGS. 6D and 6E) followed by murine CG7 (FIGS. 6A, 6D), AB11451 (FIG. 6C), chimeric HA9 (FIGS. 6B, 6E) or α-E-cadherin antibody staining (FIGS. 6A-6E). Histogram data (right) are expressed as mean fluorescent intensity from >5 fields per condition from 3 independent experiments. t-test was used for the statistical analysis. n.s.: non-significant, *p<0.05, ** p<0.01

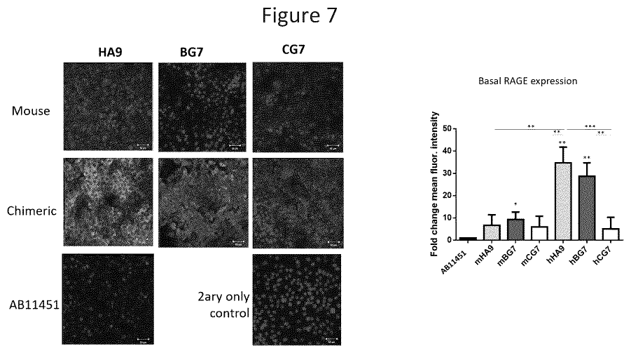

FIG. 7: hHA9 displays the highest detection of RAGE basal expression by immunofluorescent staining. HA9, BG7 and CG7 antibody clones (murine and chimeric) and AB11451 antibody were used to evaluate their ability to detect RAGE protein on SKOV3 by immunofluorescence. FITC-conjugated secondary antibodies were used to detect the primary antibodies and Hoechst to counterstain nuclei. Images were acquired on a Zeiss LSM 710 confocal microscope and mean fluorescent intensity per field was quantified as a function of number of cells using the ImageJ analysis software. (Left) Representative confocal images show hHA9 has enhanced ability to detect RAGE expression on SKOV3 cells. (Right) Histogram data are expressed as fold change compared to the average fluorescence of AB11451 obtained from >5 fields per condition from 3 independent experiments. t-test was used for the statistical analysis. * p<0.05, ** p<0.01, *** p<0.001. m=murine, h=chimeric

FIGS. 8A-8D: HA9 antibody clone has the highest uptake levels on SKOV3 cells. Cells were treated with control medium or medium containing monoclonal antibodies against RAGE at 37° C. to allow internalization for 4 h (FIG. 8A) or 24 h (FIG. 8B). After incubation, cells were washed, fixed, permeabilized, blocked and stained with fluorescent-conjugate secondary antibodies and nuclei stained with Hoechst. Alternatively, monoclonal antibodies were conjugated to a pH sensitive fluorescent-dye which fluoresced in acidic pH environments (late endosome and lysosome) (FIGS. 8C and 8D). Images were acquired on a Zeiss LSM 710 confocal microscope and mean fluorescent intensity per field was quantified as a function of number of cells using the ImageJ analysis software. (Left) Representative confocal images show hHA9 has enhanced ability to internalise on SKOV3 cells at both time points. (Right) Histogram data are expressed as fold change compared to the average fluorescence of AB11451 obtained from >5 fields per condition from 3 independent experiments. t-test was used for the statistical analysis. *p<0.05, ** p<0.01, *** p<0.001, **** p<0.0001. m=murine, h=chimeric.

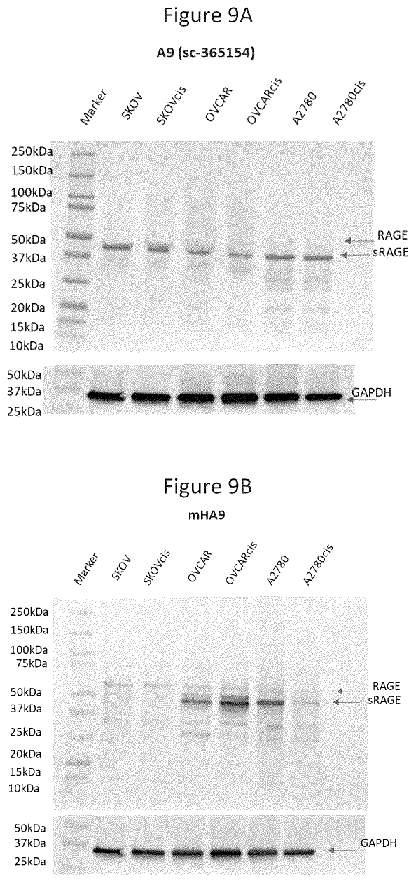

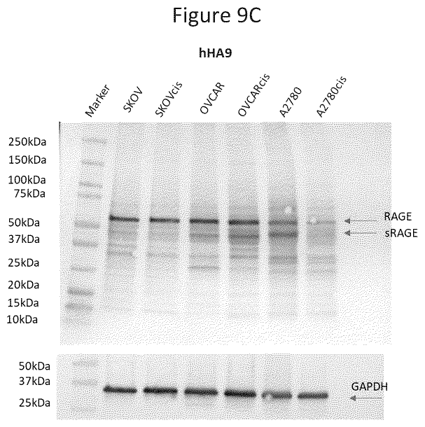

FIGS. 9A-9C: Western blot using mHA9 anti-RAGE antibody on protein extracts from OCCL. Whole-cell protein extracts were obtained from OCCL and RAGE expression was detected by immunoblot using a commercial anti-RAGE A9 antibody (Sc-365154, Santa Cruz Biotechnology) (FIG. 9A) or a murine mHA9 antibody (FIG. 9B). Blue arrow indicates full-length RAGE isoform (55 kDa) or soluble RAGE isoform (42-48 kDa). GAPDH was used as a loading control. FIG. 9C: Same protein extracts were also tested for RAGE expression levels using the hHA9 antibody confirming detection of high levels of full-length RAGE isoform (55 kDa) in all samples. GAPDH was used as a loading control.

FIGS. 10A-10D: Single dose toxicity studies using AB11551 and hHA9 auristatin F conjugates. A total of 33 female Balb-c nude mice aged 5-7 weeks and weighing approximately 20-25 g were used for the study. Animals were housed in IVC cages (up to 5 per cage) with individual mice identified by tail mark. All animals were allowed free access to a standard certified commercial diet and sanitised water during the study. For the single dose toxicity study two doses were used: 3 mg/kg and 20 mg/kg for both the AB11551 antibody conjugated to mcF (AB11451-MMAF) and the chimeric hHA9 antibody conjugated to mcF (hHA9-MMAF). Whole blood was collected via a cardiac puncture into an eppendorf tube and allowed to clot for up to 30 minutes at room temperature. Blood was centrifuged at 6000 rpm for 5 minutes and the serum fraction removed and placed in a clean eppendorf tube. Serum was snap frozen and placed in the −80° C. for subsequent aspartate aminotransferase (AST) quantification by ELISA. A commercially available ELISA kit (ab263882) was used to quantify the amount of AST present in the serum of animals sampled from all treatment groups.

FIGS. 11A-11D: Western blot using mHA9 and hHA9 anti-RAGE antibody on protein extracts from human healthy tissues. Protein extracts were obtained from human healthy tissues and RAGE expression was detected by immunoblot using commercial A9 (FIG. 11A), mHA9 (FIG. 11B) and hHA9 (FIGS. 11C and 11D) antibodies. Blue arrow indicates expected weight of full-length RAGE isoform (55 kDa). GAPDH was used as a loading control. Levels of GAPDH an inherently low in pancreas. Ovarian cancer cell lines SKOV and OVCAR were used as positive control for full length RAGE protein in FIG. 11C and FIG. 11D.

Table 1 shows the sequence information of the antibodies used in this study, including CDRs of the antibodies.

Table 2 shows V_{H} sequence information of the antibodies used in this study.

Table 3 shows V_{L} sequence information of the antibodies used in this study.

### Materials and Methods

### Murine Monoclonal Antibody Production

Murine monoclonal antibody production was performed by Bio-Rad Antibodies (formerly AbD Serotec, Bio-Rad Laboratories, Oxford, UK). All procedures were performed in accordance with the Animals (Scientific Procedures) Act 1986, and the guidance issued in ‘Responsibility in the case of Animals in Bioscience research: expectations of the major research council and charitable funding bodies.’ Monoclonal antibodies against RAGE were produced using standard protocols for monoclonal antibody production. Briefly, BALB/c mice, obtained from Charles River, Oxford, UK, were immunized with keyhole limpet hemocyanin (KLH)-conjugated RAGE, or KLH-conjugated peptides corresponding to amino acids (aa) 39-65 of the RAGE protein. Clones were selected based on a positive ELISA screen using bovine serum albumin (BSA)-conjugated peptides. Post-fusion, individual clones were then selected by limiting dilution and clonal expansion to identify genetically stable, antibody-producing cells for subsequent antibody production. 6 clones with different affinity for the full-length rRAGE were selected for antibody production (named HA9, CG7, BG7, ID9, ID7, BC3). Antibodies were purified from the tissue culture medium using protein G affinity purification.

### ELISA

A 96-well plate was coated with 5 μg/ml of RAGE synthetic peptides or RAGE recombinant protein diluted in phosphate-buffered saline (pH 7.4) overnight at 4° C. The solution was removed, washed with PBS-Tween (PBST) and the plate was blocked with ELISA ultrablock buffer (Biorad) for 1 h at room temperature. After blocking, the plate was washed with PBST and a range of concentrations of α-RAGE antibodies was applied to the plate for 2 h at room temperature. The plate was washed thoroughly with PBST and incubated with HRP-conjugated anti-mouse antibodies diluted 1:1000 in PBS-Tween. After washes with PBST, TMB Core+ substrate solution (Biorad) was added to each well and incubated at room temperature for 30 minutes. 0.2M H2SO4 was used as a stop solution. Absorbance at 450 nm was measured in a Biorad absorbance microplate reader.

### Chimeric Antibody Development and Production

Monoclonal antibody sequencing, chimeric antibody development and production was performed by Absolute Antibody Ltd (Oxford Centre for Innovation, Oxford, UK). The murine hybridomas obtained from the monoclonal antibody production described above (see Murine monoclonal antibody production section) were cultured in IMDM medium containing 10% FBS and incubated at 37° C. in a 5% CO2 environment. Total RNA was extracted from cells and a barcoded cDNA library generated through RT-PCR using a random hexamer. Next Generation Sequencing using whole transcriptome shotgun sequencing was performed on an Illumina HiSeq sequencer. Contigs were assembled using a proprietary approach and data was mined for antibody sequences identifying all viable antibody sequences (i.e. those not containing stop codons). Variable heavy and variable light domains were identified separately. The species and isotype of the identified antibody genes were confirmed. Sequences were compared with known aberrant (i.e. non-functional) antibody genes that can be present in many hybridomas and these genes were removed from analysis when necessary. The complementarity determining regions (CDRs) were identified based on the Kabat definition for CDRs. CDR identification is only performed for the primary VH and VL sequences. The amino acid sequence from those sequences with partial integrity of the DNA sequence has not been provided in this document. Once VH and VL sequences were obtained, clones including HA9, BG7 and CG7 were used to develop and produce chimeric antibodies versions. For this purpose, variable domains were designed and optimised for expression in mammalian cells (HEK293) prior to being synthesised. Sequences were then subcloned into an Absolute Antibody cloning and expression vector for the appropriate isotype and subtype of immunoglobulin heavy and light chains. HEK293 cells were transiently transfected with heavy and light chain expression vectors and cultured for 6-14 days. An appropriate volume of cells was transfected with the aim of obtaining the appropriate amount of purified antibody. Cultures were harvested and a one-step purification was performed by affinity chromatography using protein A. Upon successful purification the antibodies were buffer exchanged into PBS. The antibody was analysed for purity by SDS-PAGE and concentration determined by UV spectroscopy.

### Surface Plasmon Resonance for the Screening of Binding Kinetics of HA9 and Other α-RAGE Antibodies

For on-rate and off-rate screening, Biacore™ T200 system (Cytiva, Uppsala, Sweden) was used to perform SPR analysis with 1×HBS-EP+ buffer (0.01M Hepes, 0.15M NaCl, 0.003M ethylenediaminetetraacetic acid, and 0.05% Surfactant P20, pH 7.4) (BR100669) used as sample and running buffer. The analysis temperature at the sensor chip surface and within the compartment was set to 25° C. Firstly, pH scouting with 10 mM sodium acetate buffer 4.0 (BR-1003-49), 10 mM sodium acetate buffer 4.5 (BR-1003-50), 10 mM sodium acetate buffer 5.0 (BR-1003-51) and 10 mM sodium acetate buffer 5.5 (BR-1003-52) was performed to identify the optimal pH required for ligand (α-RAGE antibodies) immobilization. This was followed by diluting the ligand with the appropriate Acetate buffer to at least 10 μg/mL. The ligand immobilization on the surface Series S Sensor Chip CM5 (29-1496-03, Cytiva) was performed using the Amine Coupling Kit (BR100050, Cytiva) containing 1-Etyhyl-3-(3-dimethylaminopropyl)-carbodiimide hydrochloride (EDC), N-Hydroxysuccinimide (NHS) and 1.0M ethanolamine-HcL PH 8.5 in accordance with the manufacturer's instructions. For each experimental run, the ligand was immobilized in flow cells 2 or 4 while flow cells 1 or 3 were left unmodified. Flow cells 2 and 4 were used as active flow cells while flow cells 1 and 3 were used as reference flow cells. Ligand immobilization levels were typically within the range 500-900 RU for the kinetic experiments and injected with a flow rate of 10 μL/min and contact time of 300 s. Analytes (RAGE recombinant protein and synthetic peptides) were injected for 120 s with a flow rate of 30 μL/min and dissociation time of 600 s run in order of increasing concentration over the reference and active flow cells using a multi-cycle kinetic approach with five-seven concentration range. Following each binding cycle, the surface was regenerated with a 30 s injection of 10 mM Glycine-HcL, pH 2.5 regeneration solution (BR-1003-56), removing the bound antibody with a contact time of 30 s, flow rate of 30 μL/min and a stabilization period of Os. Blank cycles (buffer only injection followed by regeneration step) was also performed. Data was double referenced by first subtracting responses from the reference flow cell and then subtracting the blank cycles. Data was then fitted to a 1:1 binding model using Biacore™ T200 Evaluation Software 2.0 and Biacore™ Insight Evaluation Software.

### Immunofluorescence

To screen binding of α-RAGE antibodies by immunofluorescence, 1.5×10⁴ SKOV3 cells/well (obtained from the European Collection of Authenticated Cell Cultures, ECACC, Public Health England, UK) were seeded in 8-well chamber slides (Ibidi) in 300 μl of stripped medium and cultured for 48 h in a humidified, 5% CO2 in air atmosphere incubator at 37° C. After culture, cells were fixed with 4% paraformaldehyde (Sigma) for 20 minutes, permeabilized with 0.1% Triton X-100 (Sigma) for 10 minutes at 4° C. and blocked with 3% BSA in PBS for 1 h. Primary antibodies diluted at 4 μg/ml in 3% BSA-PBS were incubated overnight at 4° C., followed by 1 h incubation with Alexa-488 secondary antibody. Cells were washed with PBS extensively between all steps, counterstained before imaging with Hoechst (Life Technologies™) and imaged using a LSM710 confocal fluorescence microscopy system (Zeiss, UK).

Internalisation of pH-Dye Conjugated α-RAGE Antibodies

To screen internalization capacity and traffic to lysosomes of α-RAGE antibodies, all the primary antibodies used in the screening were conjugated to pHAb Amine Reactive Dye following manufacturer's instructions (Promega, UK, Cat. No. G983). This type of dye emits fluorescence when traffics to acidic environments (late endosome and lysosome). For this purpose, 1.5×104 SKOV3 cells/well were seeded in 8-well chamber slides (Ibidi) in 300 μl of stripped medium and cultured for 24 h in a humidified, 5% CO2 in air atmosphere incubator at 37° C. After culture, cells were washed in pre-warmed (37° C.) Dulbecco's phosphate buffered saline (DPBS) and slides placed on ice. Cells were treated with control medium or medium containing one of the α-RAGE antibodies conjugated to pH-dye at 10 μg/ml, and the 8-well chamber slides were incubated on ice for 30 minutes. Slides were then transferred to the incubator at 37° C. for 4 h or 24 h. After internalization time, cells were washed in DPBS, fixed in 4% paraformaldehyde for 20 min and permeabilized with 0.1% Triton X-100 (Sigma) for 10 minutes at 4° C. Cells were washed with PBS extensively between all steps, counterstained before imaging with Hoechst (Life Technologies™) and imaged using a LSM710 confocal fluorescence microscopy system (Zeiss, UK). Unconjugated α-RAGE antibodies were also used in the internalisation assay. In this case were visualized using Alexa-fluorophore secondary antibodies as described in the immunofluorescence method.

### Binding Target-Blocking Experiment Assessment by Immunofluorescence

To proof that HA9 shares similar binding epitope as other α-RAGE antibodies, an immunofluorescence approach was used. For this purpose, 1.5×10⁴ HEC1A cells/well (obtained from the European Collection of Authenticated Cell Cultures, ECACC, Public Health England, UK) were seeded in 8-well chamber slides (Ibidi) in 300 μl of stripped medium and cultured for 24 h in a humidified, 5% CO2 in air atmosphere incubator at 37° C. After culture, cells were fixed with 4% paraformaldehyde (Sigma) for 20 minutes, permeabilized with 0.1% Triton X-100 (Sigma) for 10 minutes at 4° C. and blocked with 3% BSA in PBS for 1 h. Cells were then pre-incubated with a primary antibody against RAGE at a concentration in excess (20 μg/ml in 3% BSA in PBS for 1 h) to block the specific epitope they bind, before cells were incubated with the α-RAGE antibody of interest at 4 μg/ml in 3% BSA-PBS overnight at 4° C. This was followed by 1 h incubation with Alexa-fluorophore-conjugated secondary antibodies. Cells were washed with PBS extensively between all steps, counterstained before imaging with Hoechst (Life Technologies™) and imaged using a LSM710 confocal fluorescence microscopy system (Zeiss, UK). The mean intensity of fluorescence was obtained using imageJ by measuring the integrated density per imaged field, normalised by the number of cells in the respective field and obtaining the mean from >10 fields obtained per condition. A decrease in fluorescence in the pre-blocked condition compared with the not-pre-blocked condition means both antibodies share similar epitope of binding on the same protein.

### Viability Assay

SKOV3 were seeded (500 cells/well) in 96-well tissue culture plates in 100 μl of stripped medium and cultured for 24 h in a humidified, 5% CO2 in air atmosphere incubator at 37° C. After culture, cells were treated with control medium or medium containing ADCs (0.01-50 μg/ml|), MMAF or DM1 (drug equivalent range) and incubated for 72 h. Cell growth was monitored over the 72 h h period using the RealTime-Glo™ MT Cell Viability Assay (Promega, Southampton, UK) in accordance with the manufacturer's instructions. Fluorescence was measured at 24 h intervals using a FLUOstar Omega microplate reader (BMG Labtech, Aylesbury, UK). Data was analysed to obtain the IC50 using Graphpad Prism 6.

### Western Blot (WB)

Confluent cultures of cells were lysed in ice cold RIPA buffer (Sigma-Aldrich), whole-cell protein extracts were quantified with DC protein assay, 30 μg/lane were resolved by SDS-PAGE on TGX precast gels, transferred to mini PDVF membranes, blocked 1 h at RT in 5% Bovine Serum Albumin (BSA) in TBST (Tris Buffered Saline, 0.1% Tween-20) and incubated o/n at 4° C. in the same solution with 1 μg/mL of mA9 (sc-365154, Santa Cruz Biotechnology) antibody, mHA9 antibody or hHA9 antibody, or GAPDH (Santa Cruz Biotechnology) as loading control. Membranes were then washed with TBST and incubated 1 h at RT with 0.1 μg/mL of IgG horseradish peroxidase secondary antibody (GE Healthcare) or 0.10.1 μg/mL of Goat F(ab′) 2 Anti-Mouse IgG-F(ab′) 2 horseradish peroxidase secondary antibody (Abcam). Target proteins were visualised after incubation with Clarity Western ECL Substrate and imaged using a ChemiDoc System Bio-Rad Imager. Bands were quantified with ImageLab software. Unless stated all the materials specified above were obtained from Bio-Rad Laboratories.

### Results

A synthetic peptide of the extracellular V-domain from amino acid 39 to amino acid 65 of the full-length RAGE sequence (Uniprot reference sequence: Q15109-1) was synthesized and used to obtain antibody clones; 6 clones were obtained, and denoted HA9, BG7, CG7, IG7, ID9 and BC3.

To evaluate the binding of these antibody to its target RAGE the following techniques were used:
ELISA
Binding kinetics by surface plasmon resonance (SPR) on BIAcore T200 (Cytiva, Uppsala, Sweden).
Immunofluorescence by confocal microscopy

In the evaluation of antibody binding to its target, other commercially available antibodies against RAGE were included as positive controls. The binding epitope of these antibodies are also known to be located in the V-domain of RAGE full length protein. These antibodies include AB11451 (R&D), α-RAGE N-16 (Santa CruzBiotechnology, sc-8230), α-RAGE A9 (Santa cruz Biotechnology, sc-365154) and α-RAGE Abcam (Abcam, ab37647) (FIG. 1)

HA9 Exhibits the Most Effective Binding with Synthetic RAGE Peptide and Recombinant RAGE Protein

The initial screening of the 6 murine antibody clones against RAGE was undertaken by ELISA. For this evaluation a range of 15 concentrations of each antibody clone was applied to the plate. The antigen used in this assay was the synthetic peptide (named S155) used to raise the clones (KGAPKKPPQRLEWKLNTGRTEAWKVLS) (SEQ ID NO:10). The results obtained from the ELISA showed that murine HA9 (mHA9) was the clone with highest affinity for the peptide sequence antigen (FIG. 2).

SPR analysis was subsequently used to evaluate the binding kinetics of the different murine clones to their targets (either using synthetic peptide used to produce the clones or recombinant RAGE protein (Uniprot reference: Q15109-1) Peptide S155=Original peptide from Biorad used to produce the murine clones. Human rRAGE=Full-length RAGE recombinant protein from Merck). The SPR parameters used for this analysis are described in the detailed methods. The results show that there was binding between the murine antibodies and the synthetic peptide S155 (FIGS. 3A-3C). Regarding the binding kinetics between murine clones and recombinant hRAGE, there is no interaction between any of the clones and RAGE recombinant, except for ID9 clone that shows a weak affinity (FIG. 3C).

After this preliminary screening using ELISA and BIAcore binding affinity studies the three best performing murine clones (HA9, BG7 and CG7) were engineered to produce them in chimeric humanised format, and equally assessed for binding affinity by BIACore. The commercially available AB11451 was included as a positive control._The summary of the kinetics and sensogram interpretation is that there is interaction between chimeric HA9 and the new synthetic peptides, but the affinity is weak (FIGS. 4A-4B). The binding affinity between AB11451 and the new synthetic peptide is also weak (FIGS. 4A-4B).

Given that there are commercially available antibodies that bind to similar epitopes as HA9, the binding of these commercial antibodies to the synthetic peptide and the recombinant protein was evaluated as a positive control. The commercial α-RAGE antibodies included were: α-RAGE A9 (Santa cruz Biotechnology, sc-365154) and α-RAGE Abcam (Abcam, ab37647) both binding to V-domain (as shown in FIG. 1)

The binding kinetics and sensorgram interpretation obtained with Abcam antibody show stronger affinity to RAGE recombinant and to S155 peptide compared to binding kinetics of Santacruz A9 antibody. Furthermore, Abcam antibody shows stronger affinity to RAGE recombinant than to S155 peptide. Finally, compared to HA9, Abcam antibody has stronger interaction and higher affinity to the recombinant protein and the synthetic peptide (FIG. 5).

### HA9 Shows Specific Binding to Extracellular RAGE

To further demonstrate the specificity of binding of chimeric HA9 to RAGE a binding target blocking assay was performed. For this purpose, HEC-1A cells were used to evaluate whether a pre-incubation with the N16 or Abcam antibodies resulted in a decrease of chimeric HA9 staining because of blocking of the binding epitope. AB11451, mCG7 and E-cadherin antibodies were used as controls.

The quantification of the Immunofluorescence images obtained showed that addition of N16 resulted in a statistically significant decrease of chimeric HA9 staining, supporting that N16 and hHA9 share similar or close binding epitope on RAGE. A statistically non-significant trend was also observed with AB11451. However, addition of Abcam antibody did not result in a decrease in any of the 3 antibodies (murine CG7, chimeric HA9, AB11451) (see, FIGS. 6A-6E).

These results support that chimeric HA9 binds cell surface RAGE on an epitope more similar to the N16 one than the Abcam antibody one, whereas AB11451 antibody binds to a different epitope on RAGE compared to N16 or Abcam. Supporting SPR results, murine CG7 staining was unaffected by the pre-blocking with any of the two commercial antibodies used. And as internal control, E-cadherin staining was also unaffected by the pre-blocking given that it binds to a different protein on the cell surface.

### HA9 Shows Superior Binding and Internalization in RAGE⁺ Cells

Immunofluorescent (IF) staining was used to screen the binding of the different murine and chimeric clones to RAGE protein expressed on the extracellular membrane of cancer cells in its native conformation.

Results from basal IF staining on SKOV3 cell line supports the data obtained so far: the chimeric HA9 antibody displays the most intense immunofluorescent signal, and this signal is also higher than with AB11451 antibody (FIG. 7).

Since an anti-RAGE ADC to be effective has to be internalized by the targeted cancer cells and to traffic to acidic late-endosomes and lysosomes to release the active cell-killing drug, antibody-mediated RAGE internalization was used to screen which clone had the highest uptake levels. The internalization assay was performed on SKOV3 cells treated for 4 h or 24 h with the different clones. Fluorescent secondary antibodies were then used to evaluate the amount of primary antibody taken up by SKOV3 cells (FIGS. 8A-8B). Alternatively, the clones were conjugated with a pH-dye that emits fluorescence at low pH, i.e. it confirms the traffic of conjugated antibodies to late-endosomes and lysosomes (FIGS. 8C-8D).

Results from the internalization assay shows that chimeric HA9 has the highest uptake compared with the rest of clones or with AB11451. Internalisation of pH-dye conjugated clones further confirms that chimeric HA9 has the highest uptake and traffics efficiently to acidic endosomes.

HA9 Detects Elevated Levels of RAGE Expression in Protein Extracts from Ovarian Cancer Cell Lines

Expression of RAGE full length protein (˜55 kDa) in whole-cell protein extracts from ovarian cancer cell lines (OCCL) was assessed using commercial antibody A9 (Sc-365154, FIG. 9A), as well as mHA9 antibody (FIG. 9B) and its chimeric version hHA9 antibody (FIG. 9C) by western blot (WB) technique. The cell lines included in the WB were SKOV3 (High-Grade Serous type), OVCAR3 (High-Grade Serous type), A2780 (Endometroid type), and the respective cisplatin-resistant cell lines. Anti-GAPDH was used as housekeeping protein for loading control.

The commercial A9 antibody (Sc-365154) mainly detected soluble RAGE (sRAGE, 42-48 kDa) (FIG. 9A) whereas both mHA9 antibody (FIG. 9B) and hHA9 antibody (FIG. 9C) were able to detect expression levels of full-length transmembrane RAGE (55 kDa) in all OCCL included in the study (FIGS. 9A-9C). This demonstrates that HA9 more usefully detects more isoforms of the RAGE protein.

### HA9 Exhibits Reduced Toxicity and Uptake by Healthy Cells

Single dose toxicity studies using AB11551 and hHA9 auristatin F conjugates was explored (see, FIGS. 10A-10D). The bodyweight of animals was sampled for the duration of the study for both AB11451-MMAF (FIG. 10A) and hHA9-MMAF (FIG. 10C) antibody drug conjugates. Bodyweight in animals treated with AB11451-ADC at 20 mg/kg dipped during the study, however this was not significantly different to vehicle controls (2-way ANOVA with Tukey's post-hoc test) at any time point (FIG. 10A). In the case of animals treated with hHA9-MMAF, the bodyweight for animals treated remained greater than 97% of pre-treatment levels (FIG. 10C). (FIG. 10B). AST levels were measured in serum from animals that were killed 24 h after dosing (FIG. 10B, left). AST activity in animals treated with the AB11451 conjugates at any dose tested, were not significantly different to vehicle-treated controls (ANOVA with Tukey post-hoc test). AST levels were measured in serum from animals that were killed 3 weeks after dosing (FIG. 10B, right). AST activity in animals treated with AB11451-ADC at 20 mg/kg was significantly higher than in vehicle treated controls (p=0.0096, ANOVA with Tukey post-hoc test). There was no different is levels of AST following treatment with hHA9-MMAF at 3 mg/kg or 20 mg/kg (FIG. 10D) suggesting this ADC confers a safer profile than AB11451-ADC. To explore this further, in parallel, mHA9 and hHA9 antibodies were also used to evaluate the expression of RAGE in a panel of human healthy tissues (including brain, breast, kidney, liver, lung, ovary, pancreas, spleen and uterus). WB analysis confirmed that RAGE expression evaluated with the commercial antibody A9 (Sc-365154, FIG. 11A), mHA9 (FIG. 11B) and hHA9 (FIG. 11C) antibodies. RAGE expression is detected in liver and lungs using the commercial antibody (FIG. 11A), which confirms same data as reported in the literature and public databases using alternative anti-RAGE antibodies. In the case of RAGE expression in healthy tissues detected by mHA9 or hHA9, an absent or very low expression in the healthy tissues is detected demonstrating a better antibody clone for therapeutic development.

### SUMMARY

The overall conclusion from the findings of this study is that chimeric-HA9 binds to a specific epitope on the extracellular domain of RAGE with a weaker affinity than AB11451 antibody or the commercial antibodies included in this study. However, despite the weak affinity displayed by chimeric-HA9 antibody for its intended target, its binding to RAGE protein expressed on the extracellular membrane of cells showed higher intensity and better uptake than all other antibodies including the commercially available AB11451 antibody. This data suggests that the binding of chimeric HA9 to full length RAGE in its native form on the cell surface of cells is superior.

We have generated a novel antibody targeting a single RAGE domain antigen that, despite lower binding affinity, is supremely well internalised into the cells of RAGE expressing cells, leading to increased intracellular concentrations. Further, the antibody was found to bind all tested isoforms of RAGE, reduced or no expression healthy cells compared to other antibodies and also therefore reduced cellular toxicity. Through conjugation to suitable drug cargo, use of this novel antibody will provide for effective therapeutic delivery into RAGE expressing cells, such as various cancers wherein RAGE is known to be overexpressed, permitting increased drug delivery payload and superior targetted cell killing whilst demonstrating reduced or no toxicity.

TABLE 1

| Anti- | | Binding | | | | | | |
|---|---|---|---|---|---|---|---|---|
| RAGE | | epitope/ | | | | | | |
| Clone | Proprietary/ | synthetic | | | | | | |
| name | company | peptide | CDR-H1 | CDR-H2 | CDR-H3 | CDR-L1 | CDR-L2 | CDR-L3 |
| HA9 | Own | KGAPKKP | DHWMN | QIRNRPY | YRYGLAY | RASQTID | YISESLS | QQTESWP |
| | invention | PQRLEWK | (SEQ ID | KFEKYYS | (SEQ ID | TRIH | (SEQ ID | IT |
| | | LNTGRTE | NO: 1) | DSVKG | NO: 3) | (SEQ ID | NO: 5) | (SEQ ID |
| | | AWKVLS | | (SEQ ID | | NO: 4) | | NO: 6) |
| | | (SEQ ID | | NO: 2) | | | | |
| | | NO: 10) | | | | | | |
| CG7 | Own | KGAPKKP | NYGVH | VIWTGGN | NLRGGYY | RSSQTLV | KVSNRFS | SQSTHVP |
| | invention | PQRLEWK | (SEQ ID | TDYNAAF | GAWFAY | HSNGNTY | (SEQ ID | PLT |
| | | LNTGRTE | NO: 11) | IS | (SEQ ID | LH | NO: 15) | (SEQ ID |
| | | AWKVLS | | (SEQ ID | NO: 13) | (SEQ ID | | NO: 16) |
| | | (SEQ ID | | NO: 12) | | NO: 14) | | |
| | | NO: 10) | | | | | | |
| BG7 | Own | KGAPKKP | DYWMN | QIRNRPY | YRYGLAY | RASQTID | YMSESIS | QQTNYWP |
| | invention | PQRLEWK | (SEQ ID | NFETLYS | (SEQ ID | KRIH | (SEQ ID | IT |
| | | LNTGRTE | NO: 17) | DSVKG | NO: 3) | (SEQ ID | NO: 19) | (SEQ ID |
| | | AWKVLS | | (SEQ ID | | NO: 4) | | NO: 20) |
| | | (SEQ ID | | NO: 18) | | | | |
| | | NO: 10) | | | | | | |
| ID9 | Own | KGAPKKP | TSGMGVG | HIWWDDD | SRYYGSS | N/A | N/A | N/A |
| | invention | PQRLEWK | (SEQ ID | KYYKPSL | GMDY | | | |
| | | LNTGRTE | NO: 21) | KS | (SEQ ID | | | |
| | | AWKVLS | | (SEQ ID | NO: 23) | | | |
| | | (SEQ ID | | NO: 22) | | | | |
| | | NO: 10) | | | | | | |
| IG7 | Own | KGAPKKP | DYWMN | QIRNRPY | YRYGLAY | RASQTID | YMSESIS | QQTNYWP |
| | invention | PQRLEWK | (SEQ ID | NFETLYS | (SEQ ID | KRIH | (SEQ ID | IT |
| | | LNTGRTE | NO: 17) | DSVKG | NO: 3) | (SEQ ID | NO: 19) | (SEQ ID |
| | | AWKVLS | | (SEQ ID | | NO: 4) | | NO: 20) |
| | | (SEQ ID | | NO: 18) | | | | |
| | | NO: 10) | | | | | | |
| BC3 | Own | KGAPKKP | NYGVH | VIWTGGN | NLRGGYY | N/A | N/A | N/A |
| | invention | PQRLEWK | (SEQ ID | TDYNAAF | GAWFAY | | | |
| | | LNTGRTE | NO: 11) | IS | (SEQ ID | | | |
| | | AWKVLS | | (SEQ ID | NO: 13) | | | |
| | | (SEQ ID | | NO: 12) | | | | |
| | | NO: 10) | | | | | | |
| AB11451 | R&D | GEPLVLK | N/A | N/A | N/A | N/A | N/A | N/A |
| | (MAB11451) | CKGAPKK | | | | | | |
| | | (SEQ ID | | | | | | |
| | | NO: 24) | | | | | | |

TABLE 2

| Clone | |
|---|---|
| name | Primary VH sequence |
| HA9 | EVKLVETGGGLVQPGRPMKLSCVASGFTLS |
| | DHWMNWVRQSPEKGLEWVAQIRNRPYKFEK |
| | YYSDSVKGRFTISRDDSKNSVYLQMNNLRA |
| | EDMGIYYCSSYRYGLAYWGQGTLVTVST |
| | (SEQ ID NO: 7) |
| CG7 | QVQLKQSGPGLVQPSQSLSITCTVSGFSLT |
| | NYGVHWVRQSPGKGLEWLGVIWTGGNTDYN |
| | AAFISRLSITKDNSKSQVFFKMNSLQANDT |
| | AIYYCARNLRGGYYGAWFAYWGQGTLVTVS |
| | A |
| | (SEQ ID NO: 28) |
| BG7 | EVKLDETGGGLVQPGRPMKLSCIASGFTLS |
| | DYWMNWVRQSPEKGLEWVAQIRNRPYNFET |
| | LYSDSVKGRFIISRDDSKNSVYLQMDNLGP |
| | DDSGIYYCSSYRYGLAYWGQGTLVTVSA |
| | (SEQ ID NO: 29) |
| ID9 | QVTLKESGPGILKPSQTLSLTCSFSGFSLS |
| | TSGMGVGWIRQPSGKGLEWLAHIWWDDDKY |
| | YKPSLKSQVTISKDTSRNQVFLKITSVDTA |
| | DTATYYCARSRYYGSSGMDYWGQGTSVTVS |
| | S |
| | (SEQ ID NO: 30) |
| IG7 | EVKLDETGGGLVQPGRPMKLSCIASGFTLS |
| | DYWMNWVRQSPEKGLEWVAQIRNRPYNFET |
| | LYSDSVKGRFIISRDDSKNSVYLQMDNLGP |
| | DDSGIYYCSSYRYGLAYWGQGTLVTVSA |
| | (SEQ ID NO: 29) |
| BC3 | QVQLKQSGPGLVQPSQSLSITCTVSGFSLT |
| | NYGVHWVRQSPGKGLEWLGVIWTGGNTDYN |
| | AAFISRLSITKDNSKSQVFFKMNSLQANDT |
| | AIYYCARNLRGGYYGAWFAYWGQGTLVTVS |
| | A |
| | (SEQ ID NO: 28) |

TABLE 3

| Clone | |
|---|---|
| name | Primary VL sequence |
| HA9 | DNLLTQSPAILSVSPGERVSFSCRASQTID |
| | TRIHWYQQRKNGSPRLLIRYISESLSGIPS |
| | RFSGSGSGTEFTLTINSVESEDIADYYCQQ |
| | TESWPITFGSGTKLEIK |
| | (SEQ ID NO: 8) |
| CG7 | DVVMTQTPLSLPVSLGDQASISCRSSQTLV |
| | HSNGNTYLHWYLQKPGQSPKLLIYKVSNRF |
| | SGVPDRFTGSGSGTDFTLKISRVEAEDLGV |
| | YFCSQSTHVPPLTFGAGTKLELK |
| | (SEQ ID NO: 31) |
| BG7 | DILLTQSPAILSVSPGERVSISCRASQTID |
| | KRIHWYQQRTNGSPRLLIKYMSESISGTPS |
| | RFSGSGSGTDFTLTINSVESEDIAVYYCQQ |
| | TNYWPITFGSGTKLELK |
| | (SEQ ID NO: 32) |
| ID9 | N/A |
| IG7 | DILLTQSPAILSVSPGERVSISCRASQTID |
| | KRIHWYQQRTNGSPRLLIKYMSESISGTPS |
| | RFSGSGSGTDFTLTINSVESEDIAVYYCQQ |
| | TNYWPITFGSGTKLELK |
| | (SEQ ID NO: 31) |
| BC3 | N/A |

1. An isolated antibody, or an antigen binding fragment thereof, comprising: at least one complementarity-determining region, including any combination thereof, selected from the group comprising or consisting of:
i)
CDR1:
(SEQ ID NO: 1)
DHWMN;
ii)
CDR2:
(SEQ ID NO: 2)
QIRNRPYKFEKYYSDSVKG;
iii)
CDR3:
(SEQ ID NO: 3)
YRYGLAY;
iv)
CDR4:
(SEQ ID NO: 4)
RASQTIDTRIH;
v)
CDR5:
(SEQ ID NO: 5)
YISESLS;
and
vi)
CDR6:
(SEQ ID NO: 6)
QQTESWPIT;
or
vii) an amino acid sequence having at least 70% sequence similarity with, or identity with, one of the sequences of parts i) to vi).

2. The antibody or fragment according to claim 1 comprising at least CDR:
v)
CDR5:
YISESLS (SEQ ID NO: 5).

3. The antibody or fragment according to claim 1 wherein said amino acid sequence of part vii) has 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence similarity with, or identity with, one of the sequences in parts i)-vi).

4. The antibody or fragment according to claim 1 wherein said antibody or fragment comprises a light chain variable region and a heavy chain variable region, wherein at least one of the complementarity determining regions of the heavy chain variable region and at least one of the complementarity determining regions of the light chain variable region have the following amino acid sequence:
heavy chain:
CDR1:
(SEQ ID NO: 1)
DHWMN,
CDR2:
(SEQ ID NO: 2)
QIRNRPYKFEKYYSDSVKG
or
CDR3:
(SEQ ID NO: 3)
YRYGLAY;
light chain:
CDR4:
(SEQ ID NO: 4)
RASQTIDTRIH,
CDR5:
(SEQ ID NO: 5)
YISESLS
or
CDR6:
(SEQ ID NO: 6)
QQTESWPIT;
or
an amino acid sequence having at least 70% sequence similarity or identity thereto.

5. The antibody or fragment according to claim 4 wherein the heavy chain variable region comprises a plurality, including any combination, of the following sequences, including all of the following sequences CDR1: DHWMN (SEQ ID NO: 1), CDR2: QIRNRPYKFEKYYSDSVKG (SEQ ID NO: 2) or CDR3: YRYGLAY (SEQ ID NO: 3); or an amino acid sequence having at least 70% sequence similarity or identity thereto.

6. The antibody or fragment according to claim 4 wherein the light chain variable region comprises a plurality, including any combination, of the following sequences, including all of the following sequences CDR4: RASQTIDTRIH (SEQ ID NO: 4), CDR5: YISESLS (SEQ ID NO: 5) or CDR6: QQTESWPIT (SEQ ID NO: 6), or an amino acid sequence having at least 70% sequence similarity or identity thereto.

7. The antibody or fragment according to claim 1 wherein said antibody or fragment comprises all of the following complementarity determining regions: CDR1: DHWMN (SEQ ID NO: 1), CDR2: QIRNRPYKFEKYYSDSVKG (SEQ ID NO: 2), CDR3: YRYGLAY (SEQ ID NO: 3), CDR4: RASQTIDTRIH (SEQ ID NO: 4), CDR5: YISESLS (SEQ ID NO: 5) and CDR6: QQTESWPIT (SEQ ID NO: 6).

8. The antibody or fragment according to claim 1 wherein:
i) the heavy chain variable region comprises or consists of the amino acid sequence of:
(SEQ ID NO: 7)
EVKLVETGGGLVQPGRPMKLSCVASGFTLSDHWMNWVRQSP
EKGLEWVAQIRNRPYKFEKYYSDSVKGRFTISRDDSKNSVY
LQMNNLRAEDMGIYYCSSYRYGLAYWGQGTLVTVST
(CDRs underlined);
or
an amino acid sequence having at least 70% sequence similarity or identity thereto; and/or
ii) the light chain variable region comprises or consists of the amino acid sequence:
(SEQ ID NO: 8)
DNLLTQSPAILSVSPGERVSFSCRASQTIDTRIHWYQQRKN
GSPRLLIRYISESLSGIPSRFSGSGSGTEFTLTINSVESED
IADYYCQQTESWPITFGSGTKLEIK
(CDRs underlined);
or
an amino acid sequence having at least 70% sequence similarity or identity thereto.

9. The antibody or fragment according to claim 1 wherein said antibody or fragment is:
(i) a human or humanized antibody or fragment; and/or
(ii) a chimeric antibody or fragment; and/or
(iii) a recombinant antibody or fragment.

10. A polynucleotide encoding one or more antibodies or fragments according to claim 1.

11. A vector comprising one or more polynucleotides according to claim 10.

12. A host cell comprising one or more polynucleotides according to claim 10 or one or more vectors encoding said one or more polynucleotides.

13. A pharmaceutical composition comprising the antibody or fragment(s) according to claim 1 and a pharmaceutically acceptable carrier, excipient, or diluent.

14. A combination therapeutic comprising the antibody or fragment according to claim 1 in combination with at least one other therapeutic agent.

15. The combination therapeutic according to claim 14 wherein the other therapeutic agent is an agent used to treat or prevent cancer or its associated symptoms.

16. A method of treating or preventing a disorders characterised by elevated RAGE expression in a subject in need thereof, comprising administering the antibody or fragment according to claim 1 to the subject.

17. A method of treating or preventing a disorders characterised by elevated RAGE expression in a subject in need thereof, comprising administering the pharmaceutical composition according to claim 13 to the subject.

18. A method of treating or preventing a disorder characterised by elevated RAGE expression in a subject in need thereof, comprising administering the combination therapeutic according to claim 14 to the subject.

19. The method according to claim 16 wherein said disorder is selected from diabetes, neurological disorders and cancer.

20. The method according to claim 19 wherein said cancer is selected from ovarian cancer, endometrial cancer, breast cancer and prostate cancer.


SEQUENCE LISTING (32 sequences)

SEQ ID NO: 1 - protein, 5 aa, synthetic construct
DHWMN

SEQ ID NO: 2 - protein, 19 aa, synthetic construct
QIRNRPYKFEKYYSDSVKG

SEQ ID NO: 3 - protein, 7 aa, synthetic construct
YRYGLAY

SEQ ID NO: 4 - protein, 11 aa, synthetic construct
RASQTIDTRIH

SEQ ID NO: 5 - protein, 7 aa, synthetic construct
YISESLS

SEQ ID NO: 6 - protein, 9 aa, synthetic construct
QQTESWPIT

SEQ ID NO: 7 - protein, 118 aa, synthetic construct
EVKLVETGGGLVQPGRPMKLSCVASGFTLSDHWMNWVRQSPEKGLEWVAQIRNRPYKFEKYYSDSVKGRFTISRDDSKNSVYLQMNNLRAEDMGIYYCSSYRYGLAYWGQGTLVTVST

SEQ ID NO: 8 - protein, 107 aa, synthetic construct
DNLLTQSPAILSVSPGERVSFSCRASQTIDTRIHWYQQRKNGSPRLLIRYISESLSGIPSRFSGSGSGTEFTLTINSVESEDIADYYCQQTESWPITFGSGTKLEIK

SEQ ID NO: 9 - protein, 36 aa, synthetic construct
AQNITARIGEPLVLKCKGAPKKPPQRLEWKLNTGRT

SEQ ID NO: 10 - protein, 27 aa, synthetic construct
KGAPKKPPQRLEWKLNTGRTEAWKVLS

SEQ ID NO: 11 - protein, 5 aa, synthetic construct
NYGVH

SEQ ID NO: 12 - protein, 16 aa, synthetic construct
VIWTGGNTDYNAAFIS

SEQ ID NO: 13 - protein, 13 aa, synthetic construct
NLRGGYYGAWFAY

SEQ ID NO: 14 - protein, 16 aa, synthetic construct
RSSQTLVHSNGNTYLH

SEQ ID NO: 15 - protein, 7 aa, synthetic construct
KVSNRFS

SEQ ID NO: 16 - protein, 10 aa, synthetic construct
SQSTHVPPLT

SEQ ID NO: 17 - protein, 5 aa, synthetic construct
DYWMN

SEQ ID NO: 18 - protein, 19 aa, synthetic construct
QIRNRPYNFETLYSDSVKG

SEQ ID NO: 19 - protein, 7 aa, synthetic construct
YMSESIS

SEQ ID NO: 20 - protein, 9 aa, synthetic construct
QQTNYWPIT

SEQ ID NO: 21 - protein, 7 aa, synthetic construct
TSGMGVG

SEQ ID NO: 22 - protein, 16 aa, synthetic construct
HIWWDDDKYYKPSLKS

SEQ ID NO: 23 - protein, 11 aa, synthetic construct
SRYYGSSGMDY

SEQ ID NO: 24 - protein, 14 aa, synthetic construct
GEPLVLKCKGAPKK

SEQ ID NO: 25 - protein, 18 aa, synthetic construct
AQNITARIGEPLVLKCKG

SEQ ID NO: 26 - protein, 20 aa, synthetic construct
KGAPKKPPQRLEWKLNTGRT

SEQ ID NO: 27 - protein, 21 aa, synthetic construct
AQNITARIGEPLVLKCKGAPK

SEQ ID NO: 28 - protein, 121 aa, synthetic construct
QVQLKQSGPGLVQPSQSLSITCTVSGFSLTNYGVHWVRQSPGKGLEWLGVIWTGGNTDYNAAFISRLSITKDNSKSQVFFKMNSLQANDTAIYYCARNLRGGYYGAWFAYWGQGTLVTVSA

SEQ ID NO: 29 - protein, 118 aa, synthetic construct
EVKLDETGGGLVQPGRPMKLSCIASGFTLSDYWMNWVRQSPEKGLEWVAQIRNRPYNFETLYSDSVKGRFIISRDDSKNSVYLQMDNLGPDDSGIYYCSSYRYGLAYWGQGTLVTVSA

SEQ ID NO: 30 - protein, 121 aa, synthetic construct
QVTLKESGPGILKPSQTLSLTCSFSGFSLSTSGMGVGWIRQPSGKGLEWLAHIWWDDDKYYKPSLKSQVTISKDTSRNQVFLKITSVDTADTATYYCARSRYYGSSGMDYWGQGTSVTVSS

SEQ ID NO: 31 - protein, 113 aa, synthetic construct
DVVMTQTPLSLPVSLGDQASISCRSSQTLVHSNGNTYLHWYLQKPGQSPKLLIYKVSNRFSGVPDRFTGSGSGTDFTLKISRVEAEDLGVYFCSQSTHVPPLTFGAGTKLELK

SEQ ID NO: 32 - protein, 107 aa, synthetic construct
DILLTQSPAILSVSPGERVSISCRASQTIDKRIHWYQQRTNGSPRLLIKYMSESISGTPSRFSGSGSGTDFTLTINSVESEDIAVYYCQQTNYWPITFGSGTKLELK